(19) United States
(12) Patent Application Publication
(10) Pub. No.: US 2025/0376477 A1
(43) Pub. Date: Dec. 11, 2025

(54) ANALGESIC DELTA OPIOID RECEPTOR BITOPIC LIGANDS

(71) Applicants: UNIVERSITY OF SOUTHERN CALIFORNIA, Los Angeles, CA (US); WASHINGTON UNIVERSITY, St. Louis, MO (US); UNIVERSITY OF FLORIDA RESEARCH FOUNDATION, INCORPORATED, Gainesville, FL (US); UNIVERSITY OF HEALTH SCIENCES & PHARMACY IN ST. LOUIS, St. Louis, MO (US)

(72) Inventors: Vsevolod KATRITCH, Irvine, CA (US); Saheem ZAIDI, Los Angeles, CA (US); Susruta MAJUMDAR, St. Louis, MO (US); Jay P. MCLAUGHLIN, Gainesville, FL (US); Balazs R. VARGA, St. Louis, MO (US); Tao CHE, St. Louis, MO (US)

(73) Assignees: UNIVERSITY OF SOUTHERN CALIFORNIA, Los Angeles, CA (US); WASHINGTON UNIVERSITY, St. Louis, MO (US); UNIVERSITY OF FLORIDA RESEARCH FOUNDATION, INCORPORATED, Gainesville, FL (US); UNIVERSITY OF HEALTH SCIENCES & PHARMACY IN ST. LOUIS, St. Louis, MO (US)

(21) Appl. No.: 18/876,991

(22) PCT Filed: Jun. 30, 2023
(86) PCT No.: PCT/US2023/069482

§ 371 Date: Dec. 19, 2024

Related U.S. Application Data
(60) Provisional application No. 63/357,826, filed on Jul. 1, 2022.

(51) Int. Cl. C07D 489/08; A61K 31/485

(52) U.S. Cl. CPC C07D 489/08; A61K 31/485

(57) ABSTRACT

This invention reports rational design, synthesis, and characterization of a series of a delta opioid receptor (DOR)-selective bitopic ligands targeting a conserved sodium site in DOR. The design is based on modifications in a selective DOR ligand, Naltrindole, and related indole-to-quinoline derivatives. The discovered compounds are analgesics that are free of addiction and seizure liabilities. Accordingly, a compound of the technology described herein is represented by Formula I:

### RELATED APPLICATIONS

This application claims priority under 35 U.S.C. § 119(e) to U.S. Provisional Patent Application No. 63/357,826, filed Jul. 1, 2022, which is incorporated herein by reference.

### BACKGROUND OF THE INVENTION

Selective Delta Opioid receptor (DOR) agonists are known to be effective analgesics that do not carry tolerance, addiction, and respiratory liabilities associated with morphine and other Mu opioid receptor (MOR) agonists. Successful use of DOR agonists as pain relievers, however, is precluded by strong risk of seizures, which is a key on-target side effect and liability. Targeting DOR G-protein pathways and/or partial agonists at DOR, not activating arrestin pathways, may reduce the risk of seizures.

Accordingly, there is a need for new painkillers, such as DOR agonists, that are non-additive and do not cause seizures.

### SUMMARY

This disclosure provides a compound of Formula I:

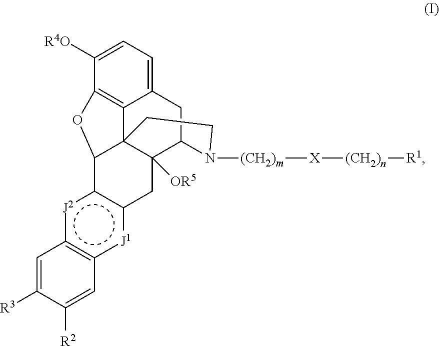

or a salt thereof;
wherein
the dotted circle of the fused monocyclic ring comprising J¹ and J² of Formula I represents aromatic bonds;
J¹ is CH or a covalent bond joining the carbon atoms adjacent to J¹;
J² is NR^{a} wherein R^{a} is a lone pair when J¹ is CH, or R^{a} is H or —(C₁-C₆)alkyl when J¹ is a covalent bond that is an aromatic bond; or
   J² is O when J¹ is the covalent bond that is an aromatic bond;
R¹ is H, —OR^{b}, —N(R^{b})₂, —(C═NR^{b})N(R^{b})₂, —NR^{b}(C═O)N(R^{b})₂, —NR^{b}(C═O)OR^{b}, —NR^{b}(C═S)N(R^{b})₂, —NR^{b}(C═NR^{b})N(R^{b})₂, —(C═NR^{b})—(C═NR^{b})N(R^{b})₂, —C═NCH₂CH₂NR^{b}, —C(═NR^{b})CH₂CH₂N(R^{b})₂, or —S(═O)₂N(R^{b})₂;
R² and R³ are each independently H, halo, OR^{b}, —N(R^{b})₂, —(C₃-C₆) cycloalkyl, or —(C₁-C₆)alkyl wherein —(C₁-C₆)alkyl is optionally substituted by halo;
each R^{b} is independently H, —(C₁-C₆)alkyl, optionally substituted phenyl, or a protecting group;
R⁴ and R⁵ are each independently H, —(C₁-C₆)alkyl, or a protecting group;
X is CH₂, O, S, NR^{b}, (C═O)NR^{b}, pyrrolyl, pyrazolyl, imidazolyl, triazolyl, pyridinyl, pyrimidinyl, pyridizinyl, pyrazinyl, or phenyl;
m is an integer from 0 to 8; and n is an integer from 0 to 8.

This disclosure also provides a method for treating pain comprising administering to a patient in need of treatment for pain an effective amount of a compound or composition of the compounds disclosed herein wherein pain sensed by the subject is reduced or alleviated.

The invention provides novel compounds of Formulas I-III, intermediates for the synthesis of compounds of Formulas I-III, as well as methods of preparing compounds of Formulas I-III. The invention also provides compounds of Formulas I-III that are useful as intermediates for the synthesis of other useful compounds. The invention provides for the use of compounds of Formulas I-III for the manufacture of medicaments useful for the treatment of pain or a pain disorder in a mammal, such as a human.

The invention provides for the use of the compositions described herein for use in medical therapy. The medical therapy can be the treatment of pain. The invention also provides for the use of a composition as described herein for the manufacture of a medicament to treat pain in a mammal. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-C. Activation of DOR (A, top), KOR (A, bottom) and MOR (B, top) in BRET assays for indole based bitopics in BRET based Gi-1 assays. DPDPE was used as reference compound for DOR, U50,488H was used as reference compound for KOR and DAMGO was used as reference compound for MOR. Four bitopics in the indole series with varying chain lengths were characterized at DOR. C5 and C6 indole were then characterized at KOR and MOR as well. While both C5/C6 showed high potency at DOR and selectivity over MOR, they retained activity at KOR as well. Activation of DOR (B, bottom), KOR (C, top) and MOR (C, bottom) in BRET assays for quinoline based bitopics in BRET based Gi-1 assays. Two bitopics in the quinoline series were characterized at all opioid subtypes. Both C5/C6 showed high potency and selectivity for DOR over other opioid subtypes.

FIG. 2. Profiling of lead bitopics at DOR at G-protein (top), βarrestin-1 (middle) and βarrestin-2 (bottom). C6 quino has lower intrinsic efficacy compared to both classical DOR agonist DPDPE as well as C5 quino in G-protein as well as at both arrestin subtypes.

FIG. 3. TRUPATH assay on DOR G protein subtypes as well as βarrestin-1 and 2, comparing potency (top) and efficacy (bottom) of C5- and C6 quino with DPDPE, TAN 67, ADL5859, Leu-Enkephalin, Deltorphin II, SNC80, SNC162 and ARM390. The bitopics C6 and C5-quino show unique functional profiling compared to known DOR orthosteric site binders.

FIG. 4A-B. C6-quino demonstrates anti-allodynic effects (A, top), driven by action on SOR (A, bottom) and thus antagonized by NTB. C6-quino exhibited dose- and time-dependent anti-allodynic activity similar to gabapentin. Mechanical allodynia induced by sciatic nerve ligation was reduced between 20 and 140 minutes after treatment with 30 mg/kg C6-quino sc. Supraspinal (icv) antinociception mediated by C6 quino is DOR dependent, yet C6-quino produced no seizures (B, top). In PK assays, C6-quino shows good systemic availability and brain penetration (B, bottom).

FIG. 5A-B. To examine locomotor (A, top) and motor coordination effects (A, bottom) and seizures (B, top), respiratory depression (B, bottom) C57BL/6J mice received various treatments (saline, vehicle, morphine, or C6-quino) via sc. Injection. Morphine treatment resulted in hyperlocomotion, while the effect of C6-quino (30 mg/kg sc.) did not significantly differ from the vehicle at any time point. In the rotarod assay, the kappa opioid agonist (KOR) U50,488H caused sedation while C6 Quino showed no propensity to cause sedation. In the seizure assay, while the canonical DOR agonist SNC80 caused seizures, C6 quino at analgesic doses showed no seizures. Morphine caused respiratory depression at the dose of 10 mg/kg. In contrast, C6-quino did not cause significant respiratory depression.

FIG. 6A-E. CryoEM structure of DOR bound to C6 and G protein heterotrimer complex. A. Overall structure of the DOR-C6-G protein heterotrimer complex. The density of C6 is shown in zoomed-in box. B. comparison of C6 binding pose with nitroindole and deltorphin. C. Conformational differences of DOR bound to C6, nitroindole, KGCHM07, and deltorphin. D. Details of interactions between C6 and orthosteric pocket. Ligand interacts with L300, W284, K214, V281, V217, H278, M132, W274, Y308, D128, I304, Q105 and Y129. E. Details of interactions between C6 and sodium pocket. Ligand side chain interacts with N131, D95, S311, S135, N310.

### DETAILED DESCRIPTION

This invention reports rational design, synthesis, and characterization of two similar series of DOR-selective bitopic ligands targeting the conserved sodium site in DOR. The design is based on modifications in the known selective DOR ligand Naltrindole (NTI) and its indole-to-quinoline derivative (Chart 1 and Chart 2).

Chart 1. Known DOR ligands and associate adverse effects (Molecules, 2020 Sep. 16; 25(18):4257 and Front Pharmacol. 2021 Nov. 3; 12:764885).

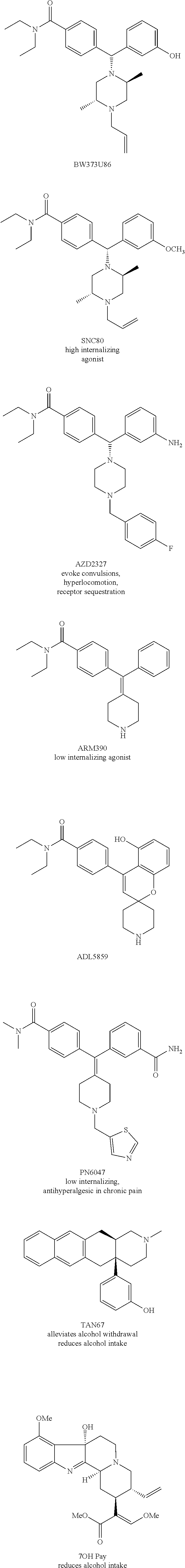

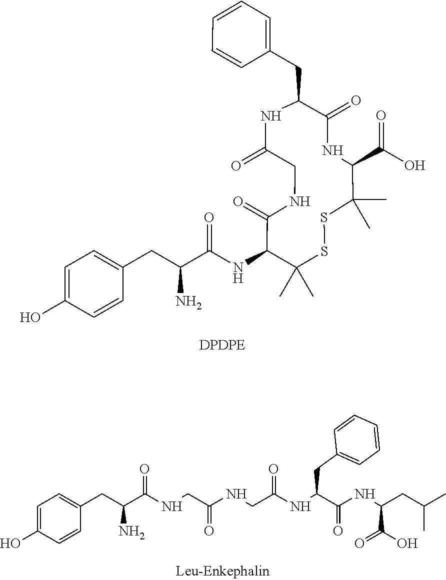 [SMILES: CC1(C)SSC(C)(C)[C@@H](NC(=O)[C@@H](N)Cc2ccc(O)cc2)C(=O)NCC(=O)N[C@@H](Cc2ccccc2)C(=O)N[C@H]1C(=O)O ; CC(C)C[C@H](NC(=O)[C@H](Cc1ccccc1)NC(=O)CNC(=O)CNC(=O)[C@@H](N)Cc1ccc(O)cc1)C(=O)O]

Chart 2. Design of DOR bitopics across three cores.

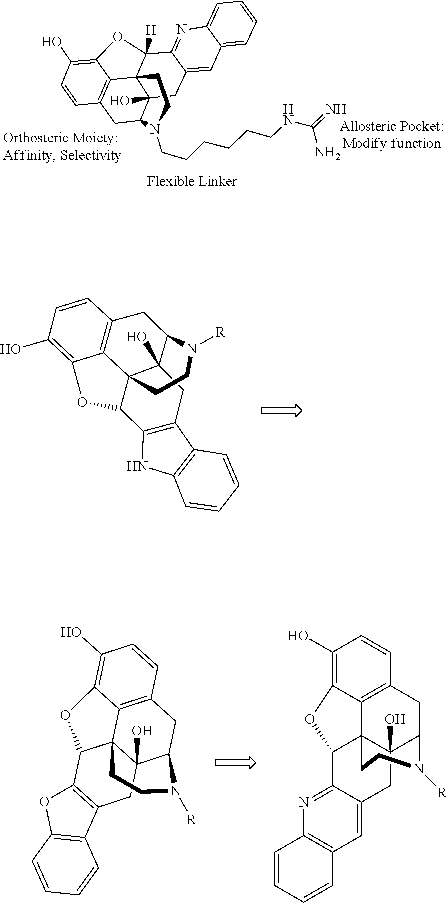

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability, necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number 10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patients may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

An “effective amount” also refers to an amount effective to bring about a recited effect, such as an amount necessary to form products in a reaction mixture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (1-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at a carbon atom or two different carbon atoms.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazepane, 1,4-diazepane, 1,4-oxazepane, and 1,4-oxathiepine. The group may be a terminal group or a bridging group.

The term “aromatic” refers to either an aryl or heteroaryl group or substituent described herein. Additionally, an aromatic moiety may be a bisaromatic moiety, a trisaromatic moiety, and so on. A bisaromatic moiety has a single bond between two aromatic moieties such as, but not limited to, biphenyl, or bipyridine. Similarly, a trisaromatic moiety has a single bond between each aromatic moiety.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below. For example, a phenyl moiety or group may be substituted with one or more substituents RX where RX is at the ortho-, meta-, or para-position, and X is an integer variable of 1 to 5.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidazolyl, indazolyl, indolizinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The terms “racemic mixture” and “racemate” refer to an equimolar mixture of two enantiomeric species, devoid of optical activity.

The term “IC₅₀” is generally defined as the concentration required to inhibit a specific biological or biochemical function by half, or to kill 50% of the cells in a designated time period, typically 24 hours.

### EMBODIMENTS OF THE TECHNOLOGY

### This Disclosure Provides a Compound of Formula I:

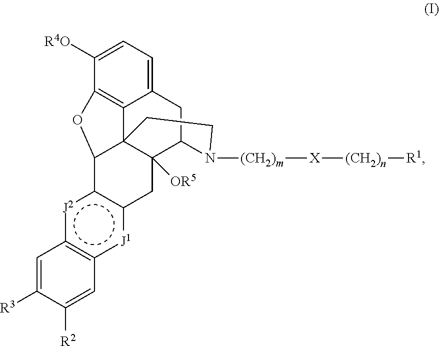

or a salt thereof;
wherein
the dotted circle of the fused monocyclic ring comprising J¹ and J² of Formula I represents aromatic bonds;
J¹ is CH or a covalent bond joining the carbon atoms adjacent to J¹;
J² is NR^{a} wherein R^{a} is a lone pair when J¹ is CH, or R^{a} is H or —(C₁-C₆)alkyl when J¹ is a covalent bond that is an aromatic bond; or
   J² is O when J¹ is the covalent bond that is an aromatic bond;
R¹ is H, —OR^{b}, —N(R^{b})₂, —(C═NR^{b})N(R^{b})₂, —NR^{b}(C═O)N(R^{b})₂, —NR^{b}(C═O)OR′, —NR^{b}(C═S)N(R^{b})₂, —NR^{b}(C═NR^{b})N(R^{b})₂, —(C═NR^{b})—(C═NR^{b})N(R^{b})₂, —C═NCH₂CH₂NR^{b}, —C(═NR^{b})CH₂CH₂N(R^{b})₂, or —S(═O)₂N(R^{b})₂;
R² and R³ are each independently H, halo, OR^{b}, —N(R^{b})₂, —(C₃-C₆) cycloalkyl, or —(C₁-C₆)alkyl wherein —(C₁-C₆)alkyl is optionally substituted by halo;
each R^{b} is independently H, (C₁-C₆)alkyl, optionally substituted phenyl, or a protecting group;
R⁴ and R⁵ are each independently H, —(C₁-C₆)alkyl, or a protecting group;
X is CH₂, O, S, NR^{b}, (C═O)NR^{b}, pyrrolyl, pyrazolyl, imidazolyl, triazolyl, pyridinyl, pyrimidinyl, pyridizinyl, pyrazinyl, or phenyl;
   m is an integer from 0 to 8; and n is an integer from 0 to 8.

In some embodiments, the compound of Formula I is a compound of Formula II:

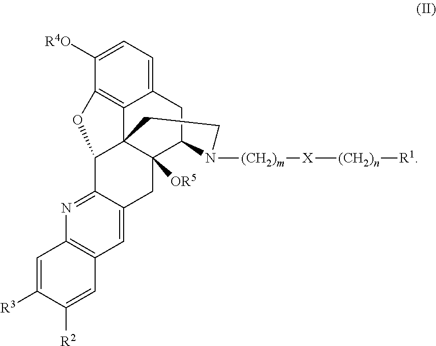

In some embodiments, the compound of Formula I is a compound of Formula III:

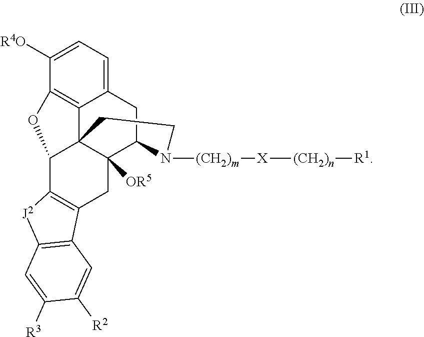

In some embodiments, J² is NR^{a}. In some embodiments, J² is O. In some embodiments, R¹ is —NR^{b}(C═NR^{b})N(R^{b})₂. In some embodiments, R² and R³ are each independently chloro, trifluoromethyl, isopropyl, tert-butyl, cyclopropyl, methoxy, or ethoxy. In some embodiments, R⁴ and R⁵ are H. In some embodiments, R^{b} is H. In some embodiments, the protecting group is the moiety tert-butyloxycarbonyl.

### In Some Embodiments, X is:

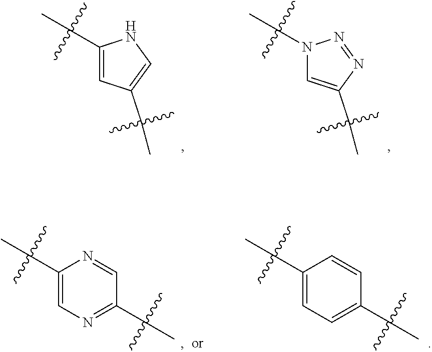 [SMILES: *c1c[nH]c(*)c1 ; *c1cn(*)nn1 ; *c1cnc(*)cn1 ; *c1ccc(*)cc1]

In some embodiments, X is CH₂. In some embodiments, m is 0, 1, 2, 3, 4, 5, 6, 7, or 8. In some embodiments, n is 0, 1, 2, 3, 4, 5, 6, 7, or 8.

### In Some Embodiments, the Compound is:

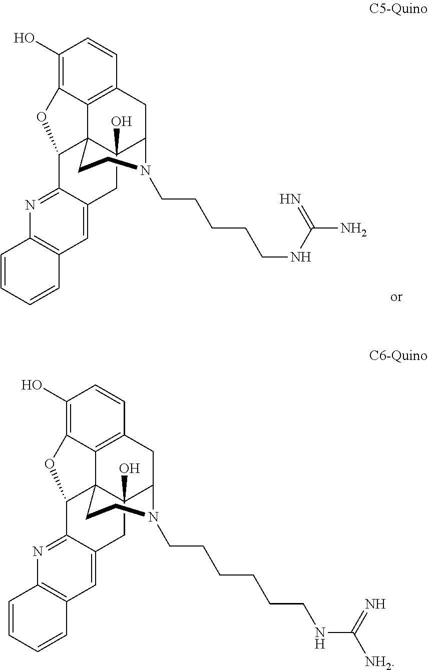

### In Other Embodiments, the Compound is:

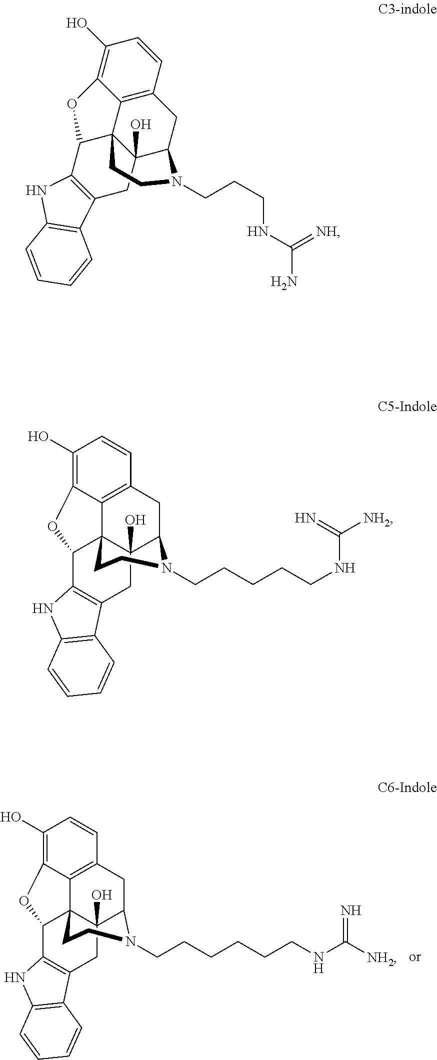

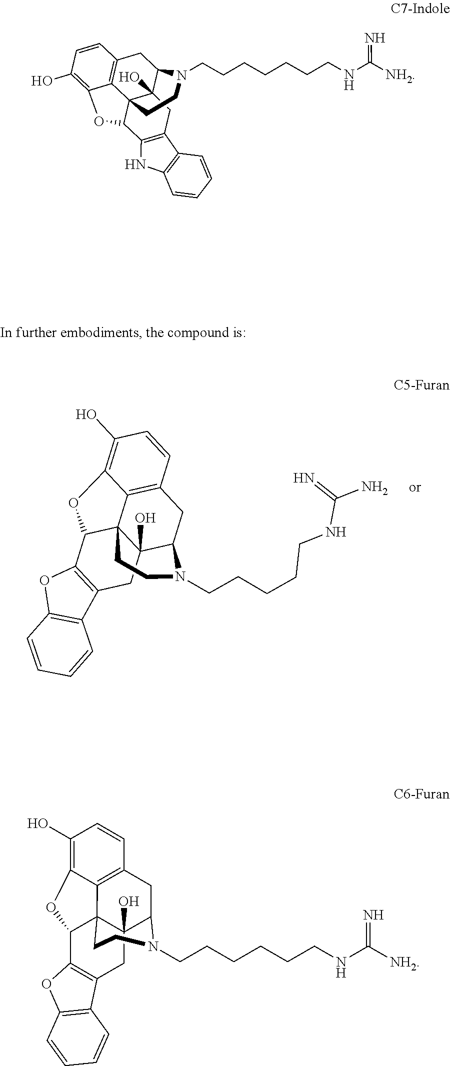

This disclosure also provides a pharmaceutical composition comprising a compound disclosed herein and an excipient.

Additionally, this disclosure provides a method for treating pain comprising administering to a patient in need of treatment for pain an effective amount of a compound or composition disclosed herein, wherein pain sensed by the subject is reduced or alleviated.

The patient receiving therapy can be currently experiencing pain or may experience intermittent bouts of pain. In some embodiments, an effective amount of a compound described herein is administered via a medicament in a single dose or by multiple doses. In some embodiments, the medicament is administered once per day, twice per day, three times per day, 4 times per day, or multiple times per day. In some embodiments, the amount of the compound in the medicament is based on the body weight of the patient. In some embodiments, the effective amount of the compound is about 1 mg/kg to about 50 mg/kg. In some embodiments, the effective amount of the compound is about 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, or 100 mg/kg, or a multiple thereof (e.g., 1×, 2×, or 3×), depending on the severity of the paid.

Furthermore, this disclosure provides the use of a compound or composition disclosed herein for the preparation of a medicament for the treatment of pain; or the use of a compound or composition disclosed herein for the treatment of pain.

### EMBODIMENTS OF THE INVENTION

1. A Compound of Formula I:

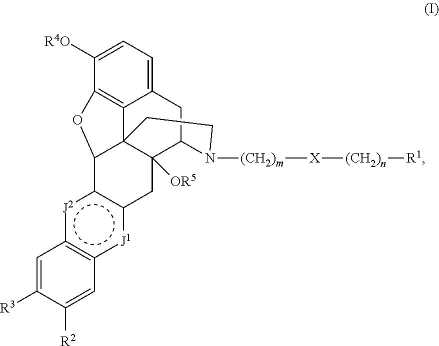

or a salt thereof;
wherein
the dotted circle of the fused monocyclic ring comprising J¹ and J² of Formula I represents aromatic bonds;
J¹ is CH or an aromatic bond joining the carbon atoms adjacent to J¹;
J² is NR^{a} wherein R^{a} is a lone pair when J¹ is CH, or R^{a} is H or —(C₁-C₆)alkyl when J¹ is an aromatic bond; or J² is O when J¹ is an aromatic bond;
R¹ is —NR^{b}(C═NR^{b})N(R^{b})₂, —OR^{b}, —N(R^{b})₂, —(C═NR^{b})N(R^{b})₂, —NR^{b}(C═O)N(R^{b})₂, —NR^{b}(C═O)OR^{b}, —NR^{b}(C═S)N(R^{b})₂, —C═NCH₂CH₂NR^{b}, —C(═NR^{b})CH₂CH₂N(R^{b})₂, or —S(═O) 2N(R^{b})₂;
R² and R³ are each independently H, halo, —OR^{b}, —N(R^{b})₂, —(C₃-C₆) cycloalkyl, or —(C₁-C₆)alkyl wherein —(C₁-C₆)alkyl is optionally substituted by halo;
each R^{b} is independently H, —(C₁-C₆)alkyl, optionally substituted phenyl, or a protecting group;
R⁴ and R⁵ are each independently H, —(C₁-C₆)alkyl, or a protecting group;
X is CH₂, O, S, NR^{b}, (C═O)NR^{b}, pyrrolyl, pyrazolyl, imidazolyl, triazolyl, pyridinyl, pyrimidinyl, pyridizinyl, pyrazinyl, or phenyl;
m is an integer from 0 to 8; and n is 4, 5, or an integer from 0 to 8.

### 2. The Compound of Embodiment 1 Represented by Formula II:

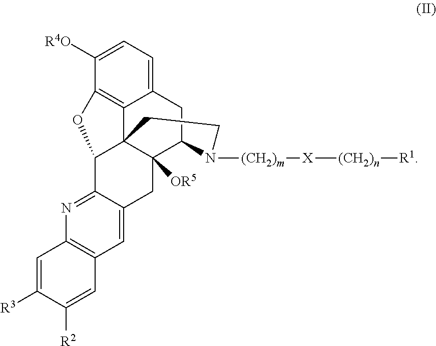

3. The Compound of Embodiment 1 Represented by Formula III:

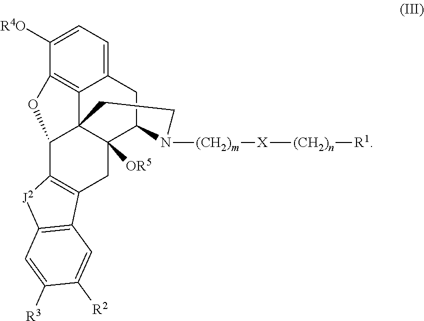

4. The Compound of Embodiments 1 or 3 Wherein J² is NR^{a}.

5. The compound of embodiments 1 or 3 wherein J² is O.

6. The compound of any one of embodiments 1-5 wherein R¹ is —NR^{b}(C═NR^{b})N(R^{b})₂.

7. The compound of any one of embodiments 1-6 wherein R² and R³ are each independently H, chloro, trifluoromethyl, isopropyl, tert-butyl, cyclopropyl, methoxy, or ethoxy.

8. The compound of any one of embodiments 1-7 wherein R⁴ and R⁵ are H.

9. The compound of any one of embodiments 1-8 wherein R^{b} is H.

10. The compound of any one of embodiments 1-9 wherein X is CH₂.

11. The compound of any one of embodiments 1-9 wherein X is:

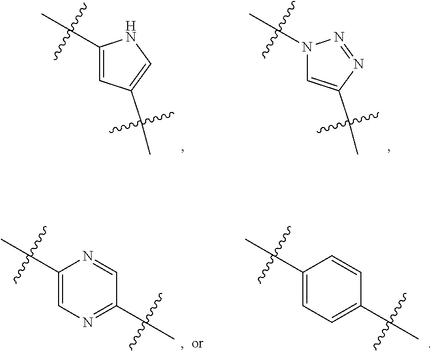 [SMILES: *c1c[nH]c(*)c1 ; *c1cn(*)nn1 ; *c1cnc(*)cn1 ; *c1ccc(*)cc1]

12. The compound of embodiment 1 or 2 wherein the compound is:

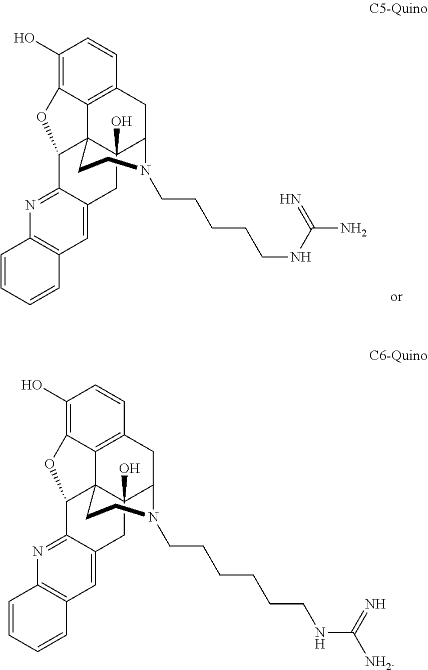

13. The compound of embodiment 1 or 3 wherein the compound is:

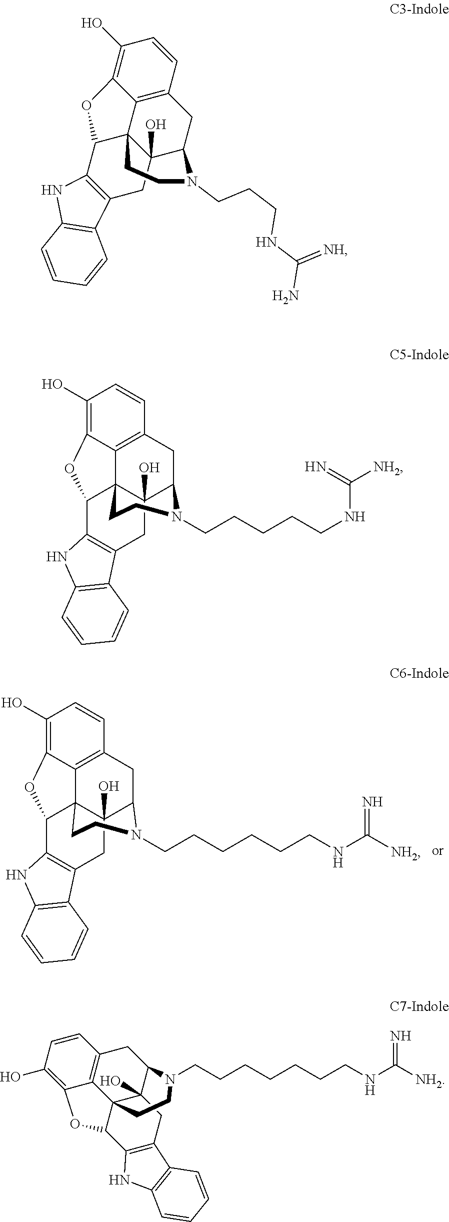

14. The compound of embodiment 1 or 3 wherein the compound is:

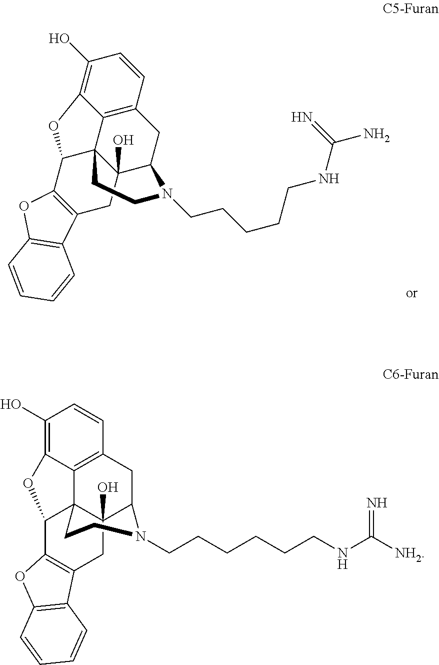

15. A method for treating pain wherein the method comprises administering to a patient having pain an effective amount of a compound or a composition of any one of embodiments 1-14 wherein pain sensed by the subject is thereby reduced or alleviated.

### Results and Discussion

Selective Delta Opioid receptor (DOR) agonists are known to be effective analgesics that do not carry tolerance, addiction, and respiratory liabilities associated with morphine and other Mu opioid agonists. Successful use of DOR agonists as painkillers, however, is precluded by strong risk of seizures, which is a key on-target side effect and liability. Ligands targeting DOR G-protein pathways, but not arrestin pathways, may reduce the risk of seizures. This disclosure reports rational design, synthesis, and characterization of two similar series of DOR-selective bitopic ligands targeting the conserved sodium site in DOR. The design is based on modifications in know selective DOR ligand Naltrindole and its indole-to-quinoline derivative (Chart 3).

Biochemical assays and cryo-EM structures show that bitopic ligands with C5-guano and C6-guano extensions have the best DOR affinity and potency in G-protein pathways, but C6-guano compounds have drastically reduced arrestin signaling. In vivo, C6-guano shows dose-dependent analgesia in subcutaneous and oral administration, comparable to analgesia conferred by standard of care gabapentin. Unlike classical DOR agonists, subjects receiving C6-guano compounds at a high dose completely lacked seizures. Unlike the MOR agonist morphine, C6-guano did not impact locomotor activity or respiration in mice. The compounds have high bioavailability and brain penetration. They are therefore strong clinical candidates.

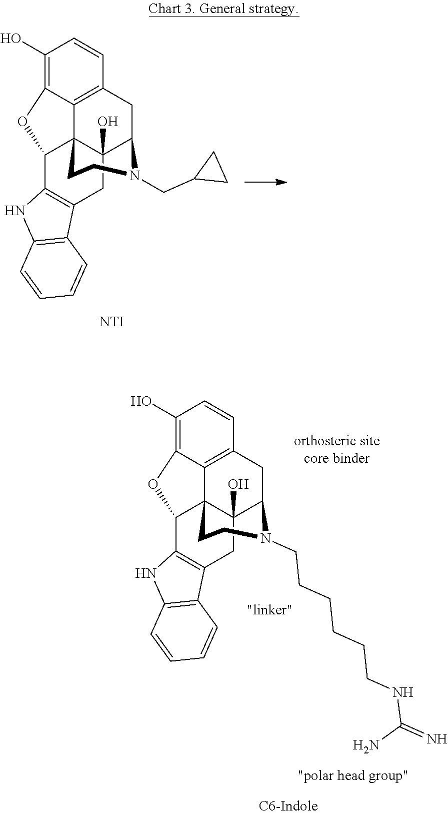

### GENERAL SYNTHETIC METHODS

The invention also relates to methods of making the compounds and compositions of the invention. The compounds and compositions can be prepared by any of the applicable techniques of organic synthesis, for example, the techniques described herein. Many such techniques are well known in the art. However, many of the known techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6, Michael B. Smith; as well as standard organic reference texts such as March's Advanced Organic Chemistry: Reactions. Mechanisms, and Structure, 5^{th} Ed. by M. B. Smith and J. March (John Wiley & Sons, New York, 2001), Comprehensive Organic Synthesis: Selectivity, Strategy & Efficiency in Modern Organic Chemistry, in 9 Volumes, Barry M. Trost, Ed.-in-Chief (Pergamon Press, New York, 1993 printing)); Advanced Organic Chemistry, Part B: Reactions and Synthesis, Second Edition, Cary and Sundberg (1983); Protecting Groups in Organic Synthesis. Second Edition, Greene, T. W., and Wutz, P. G. M., John Wiley & Sons, New York; and Comprehensive Organic Transformations, Larock, R. C., Second Edition, John Wiley & Sons, New York (1999).

A number of exemplary methods for the preparation of the compounds of the invention are provided below. These methods are intended to illustrate the nature of such preparations are not intended to limit the scope of applicable methods.

Generally, the reaction conditions such as temperature, reaction time, solvents, work-up procedures, and the like, will be those common in the art for the particular reaction to be performed. The cited reference material, together with material cited therein, contains detailed descriptions of such conditions. Typically, the temperatures will be −100° C. to 200° C., solvents will be aprotic or protic depending on the conditions required, and reaction times will be 1 minute to 10 days. Work-up typically consists of quenching any unreacted reagents followed by partition between a water/organic layer system (extraction) and separation of the layer containing the product.

Oxidation and reduction reactions are typically carried out at temperatures near room temperature (about 20° C.), although for metal hydride reductions frequently the temperature is reduced to 0° C. to −100° C. Heating can also be used when appropriate. Solvents are typically aprotic for reductions and may be either protic or aprotic for oxidations. Reaction times are adjusted to achieve desired conversions.

Condensation reactions are typically carried out at temperatures near room temperature, although for non-equilibrating, kinetically controlled condensations reduced temperatures (0° C. to −100° C.) are also common. Solvents can be either protic (common in equilibrating reactions) or aprotic (common in kinetically controlled reactions). Standard synthetic techniques such as azeotropic removal of reaction by-products and use of anhydrous reaction conditions (e.g., inert gas environments) are common in the art and will be applied when applicable.

Protecting Groups. The term “protecting group” refers to any group which, when bound to a hydroxy or other heteroatom prevents undesired reactions from occurring at this group and which can be removed by conventional chemical or enzymatic steps to reestablish the hydroxyl group. The particular removable protecting group employed is not always critical and preferred removable hydroxyl blocking groups include conventional substituents such as, for example, allyl, benzyl, acetyl, chloroacetyl, thiobenzyl, benzylidene, phenacyl, methyl methoxy, silyl ethers (e.g., trimethylsilyl (TMS), 1-butyl-diphenylsilyl (TBDPS), or 1-butyldimethylsilyl (TBS)) and any other group that can be introduced chemically onto a hydroxyl functionality and later selectively removed either by chemical or enzymatic methods in mild conditions compatible with the nature of the product.

Suitable hydroxyl protecting groups are known to those skilled in the art and disclosed in more detail in T. W. Greene, Protecting Groups In Organic Synthesis; Wiley: New York, 1981 (“Greene”) and the references cited therein, and Kocienski, Philip J.; Protecting Groups (Georg Thieme Verlag Stuttgart, New York, 1994), both of which are incorporated herein by reference.

Protecting groups are available, commonly known and used, and are optionally used to prevent side reactions with the protected group during synthetic procedures, i.e., routes or methods to prepare the compounds by the methods of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis.

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention provides therapeutic methods of treating pain in a mammal, which involve administering to a mammal having cancer an effective amount of a compound or composition described herein. A mammal includes a primate, human, rodent, canine, feline, bovine, ovine, equine, swine, caprine, bovine and the like.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

Chemistry. Reagents purchased from Alfa Aesar, Fisher Scientific and Sigma-Aldrich Chemicals were used without further purification. Reaction mixtures were purified by silica gel flash chromatography on E. Merck 230-400 mesh silica gel 60 using a Teledyne ISCO CombiFlash Rf instrument with UV detection at 280 and 254 nm. RediSep Rf silica gel normal phase columns were used with MeOH in DCM or EtOAc in Hexane solvent systems with gradients as indicated. Reverse phase RediSep columns (C18, 100 Å, 5 micron) were used with H₂O and MeCN containing 0.05% TFA. Reported yields are isolated yields upon purification of each intermediate. Final clean (purity ≥95%, LC-MS Agilent 1100 Series LC/MSD) compounds were used for the study. NMR spectra were collected using Varian 400 MHz NMR instrument at the NMR facility of Washington University School of Medicine in St. Louis. Chemical shifts are reported in parts per million (ppm) relative to residual solvent peaks at the nearest 0.01 for proton and 0.1 for carbon: CDCl₃ ¹H: 7.26, ¹³C: 77.1; and CD₃OD ¹H: 3.31, ¹³C: 49.0). Peak multiplicity in NMR spectra are apparent peaks as reported by MestreNova software, namely s—singlet, d—doublet, t—triplet, q—quartet, m—multiplet for example. Coupling constant (J) values are expressed in Hz. Mass spectra were obtained at the St. Louis College of Pharmacy using the Agilent 1100 Series LC/MSD by electrospray (ESI) ionization with a gradient elution program (Ascentis Express Peptide C18 column, acetonitrile/water 5/95/95/5, 5 min, 0.05% formic acid) and UV detection (214 nM/254 nM). High resolution mass spectra were obtained using a Bruker 10 T APEX-Qe FTICR-MS and the accurate masses are reported for the molecular ion [M+H]⁺. Detailed experimental descriptions and characterization of the new compounds are included in the synthesis section.

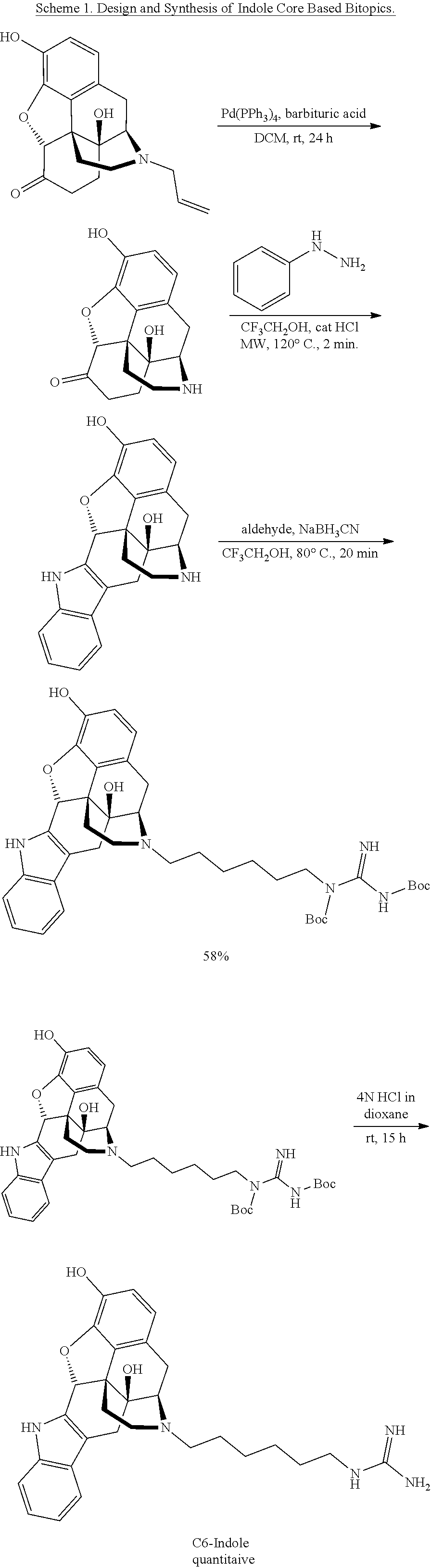

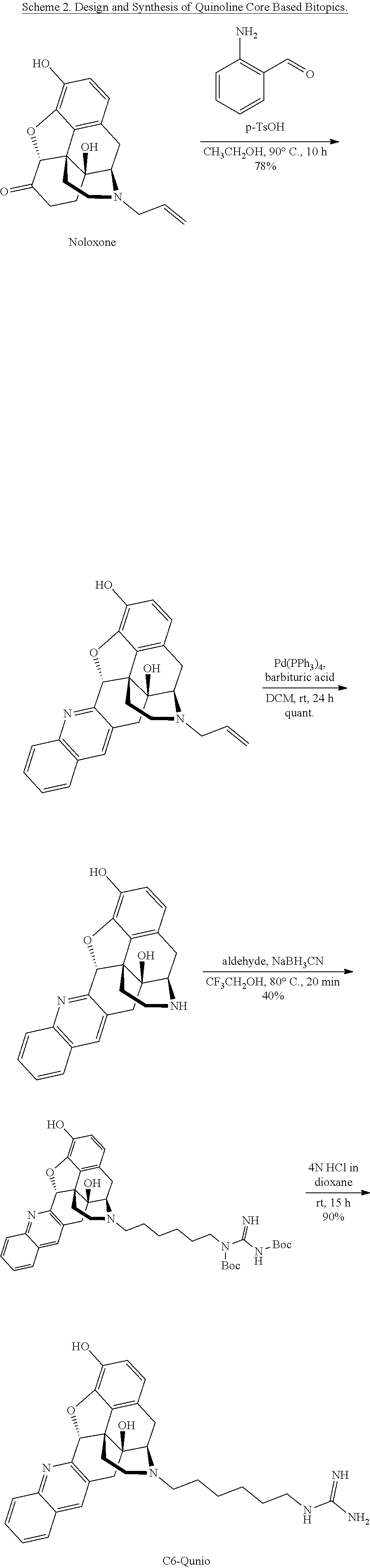

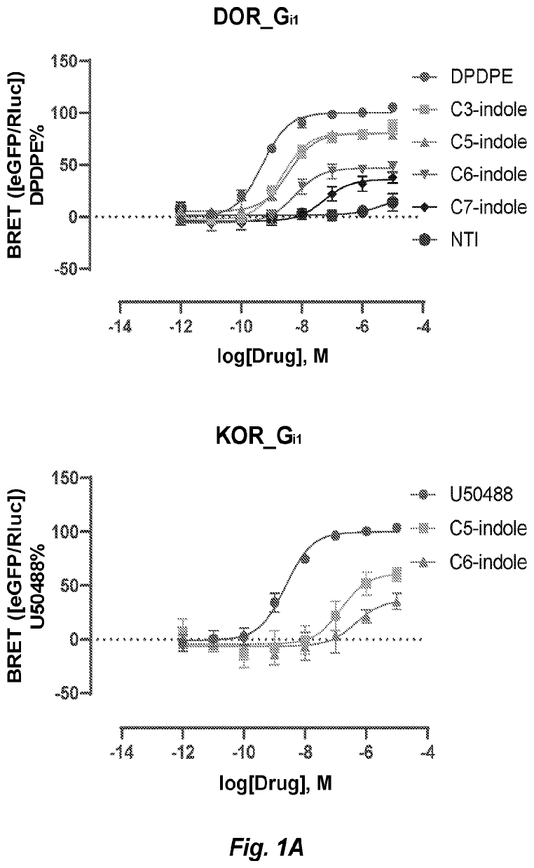

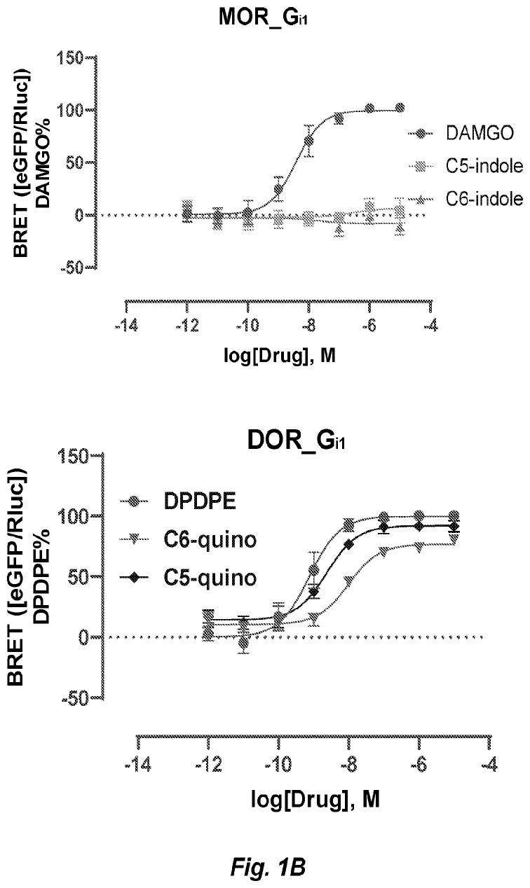

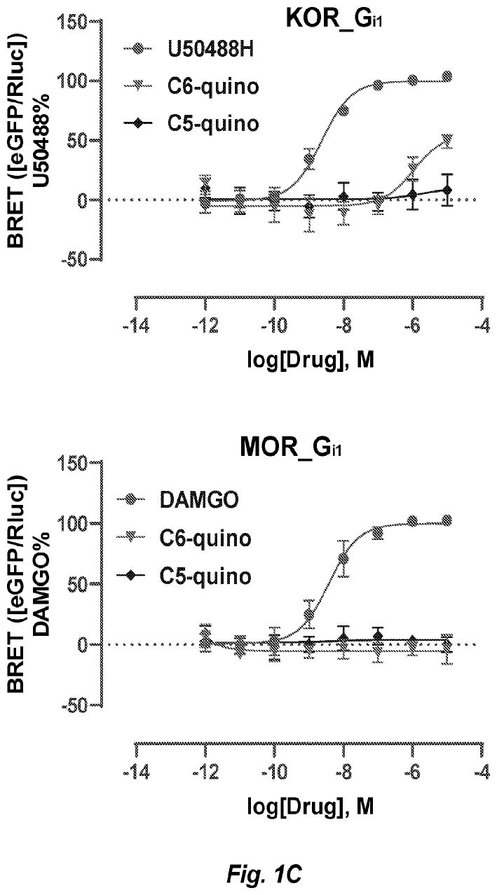

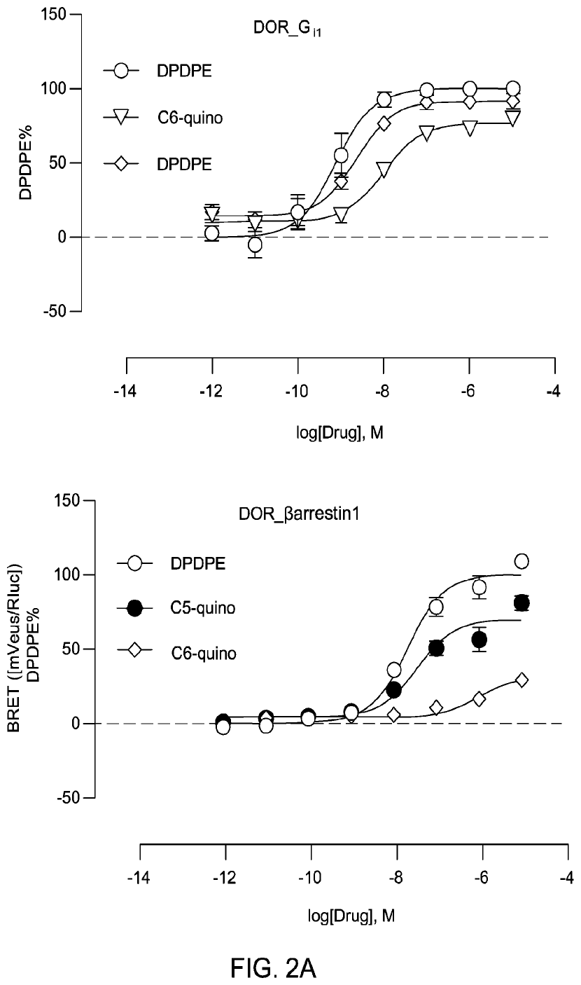

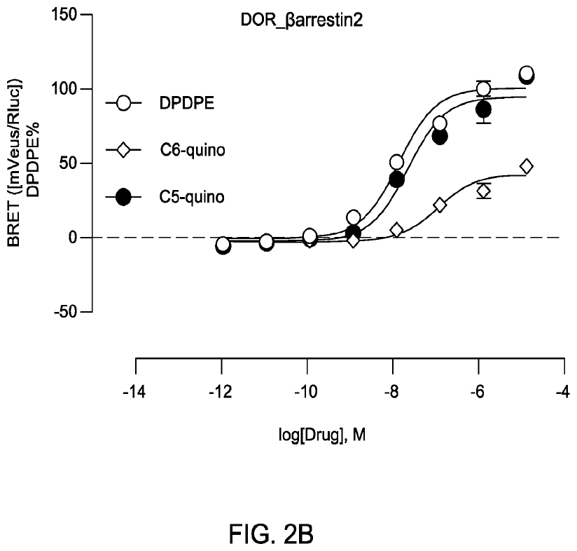

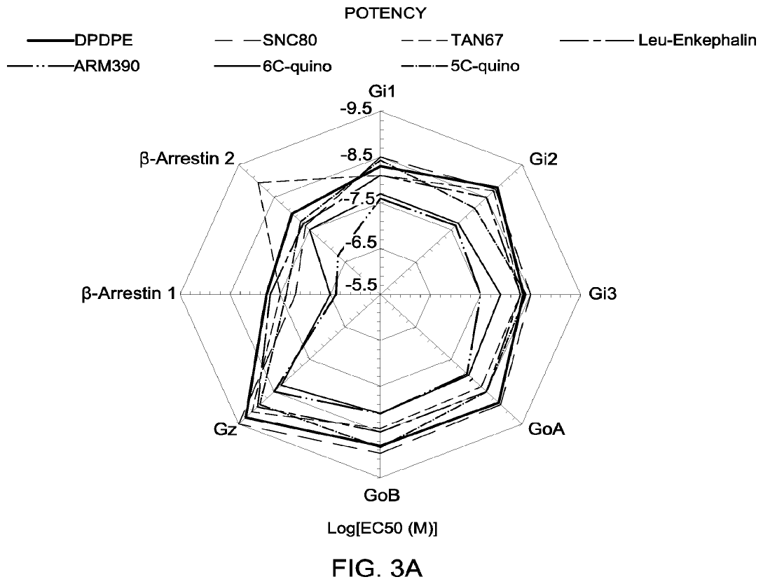

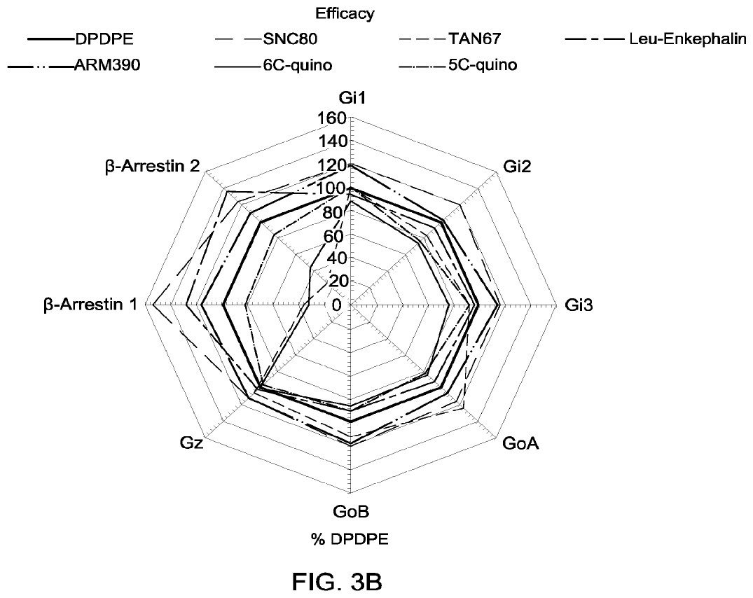

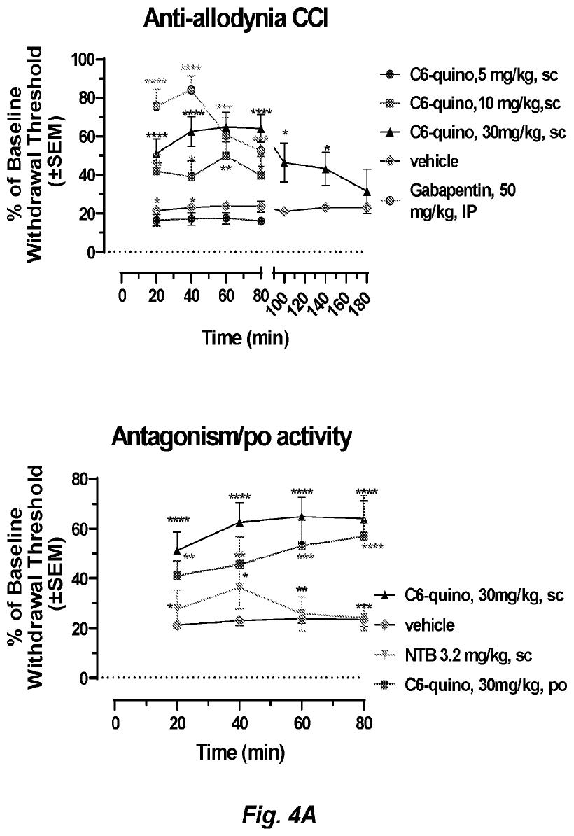

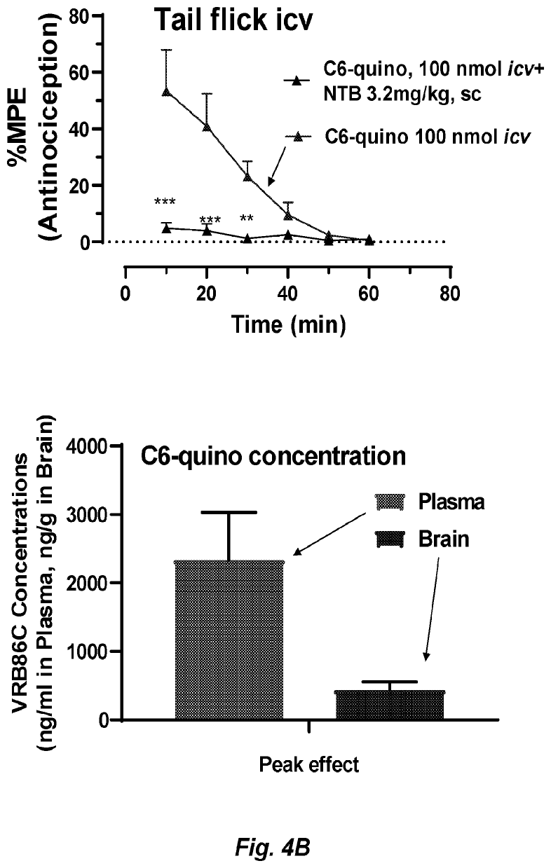

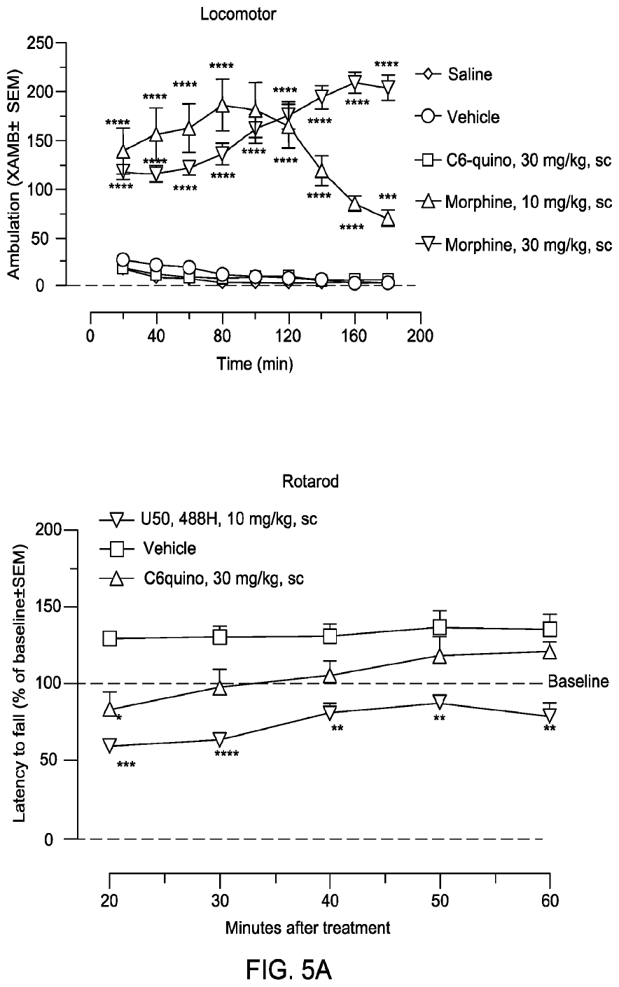

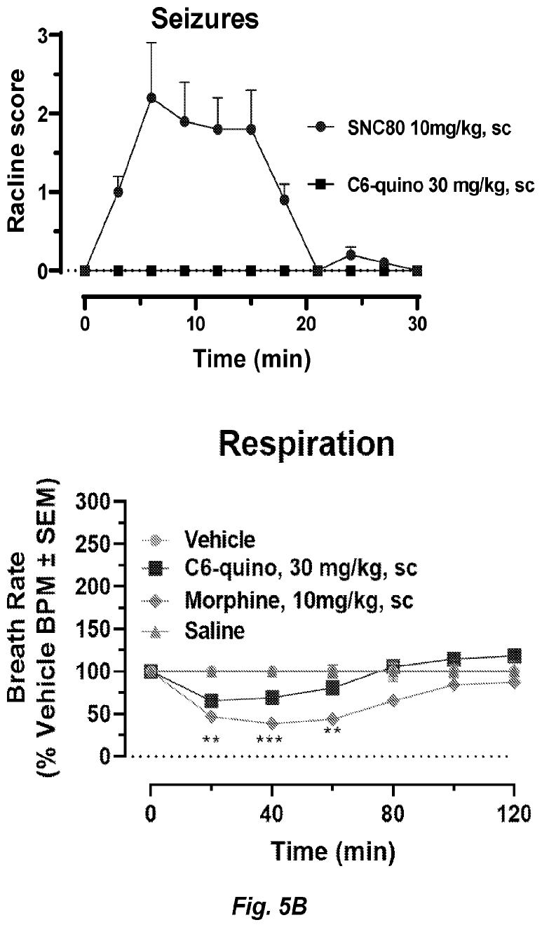

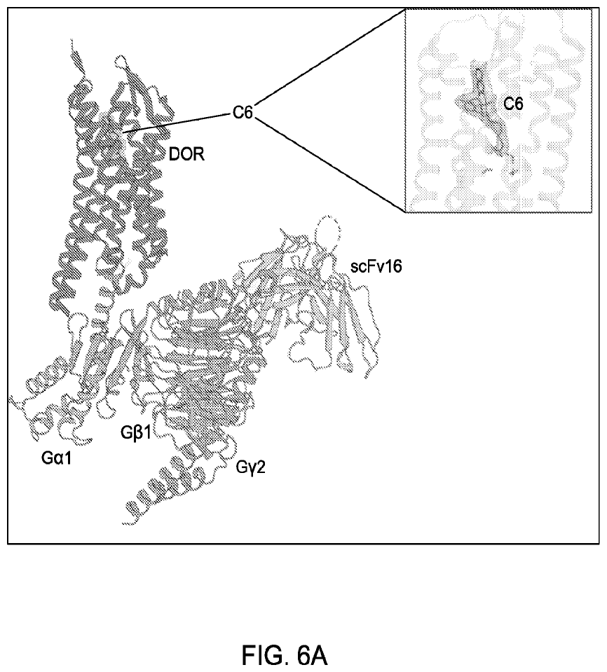

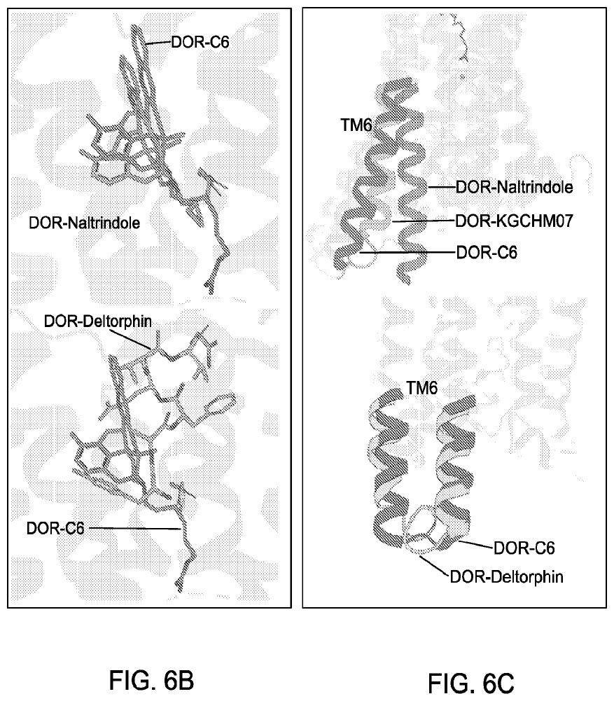

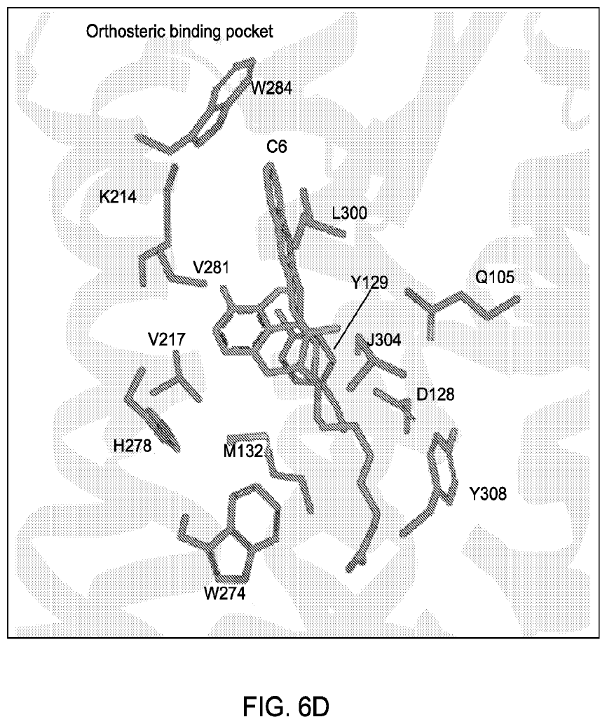

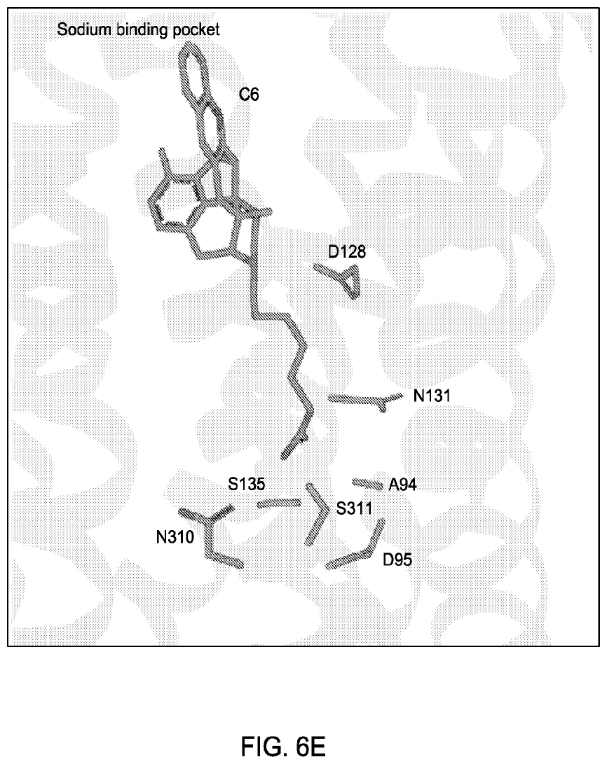

Various compounds of Formula I, for example, the Scheme 1 and Scheme 2 compounds C6-Indole and C6-Quino, were found to be orally active, G-protein biased, effective in chronic pain models, and do not cause seizures (or have a rate of seizure occurrence far less than known DOR agonists). See Table 1 and FIGS. 1-5 for relevant data.

TABLE 1
Druglike Properties.

| ADMET studies | Acceptable metrics | 6C-Quino |
|---|---|---|
| MW/ACD LogD7.4/TPSA | <500/<5/<90A | 515 g/mol, 127 |
| Plasma protein binding | >5% free | 12% |
| Microsomal stability | >10% at 4 h or > 2 h t_{1/2} | >2 h t_{1/2} |
| PK-brain:plasma, t_{1/2} | >2 h | 1:6, 2 h |
| n HBD | <5 | 4 |
| n HBA | <10 | 7 |
| logP | <5 | 3.5 |
| Orally active | YES | YES |

### Example 2. Synthesis of Bitopic Cores and Characterization

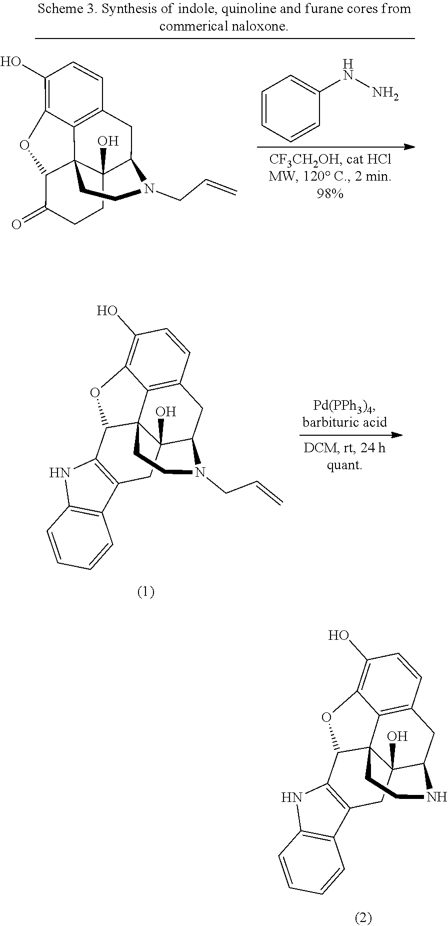

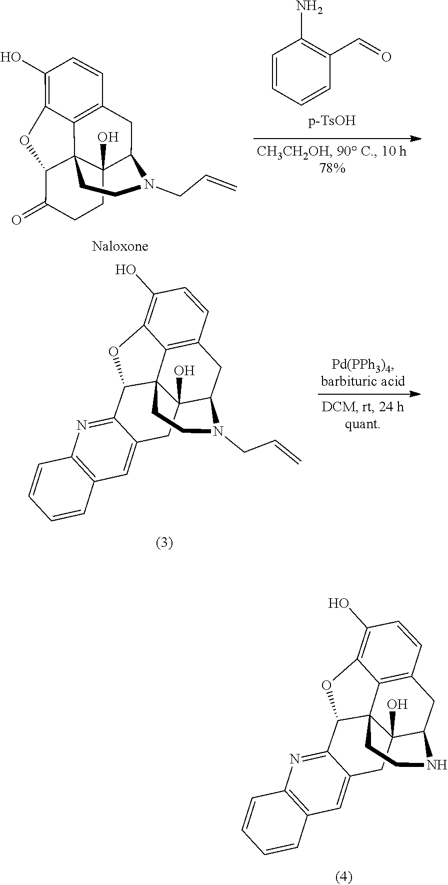

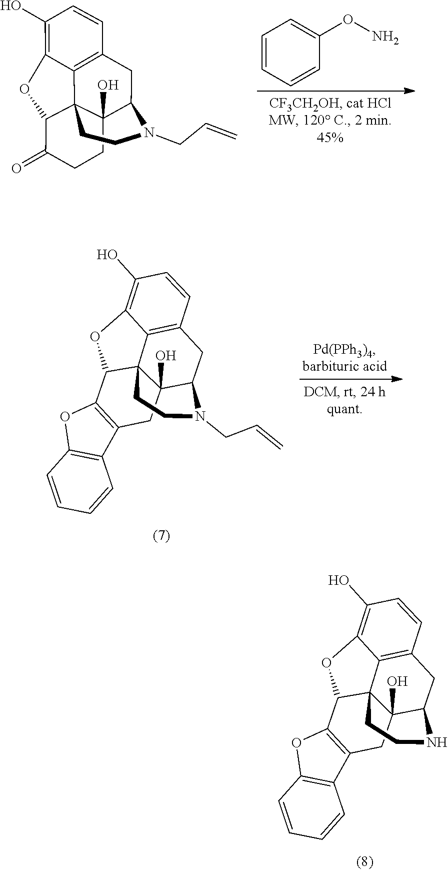

### Scheme 4. Synthesis of Boc-Protected Aldehydes (DBPC═N,N′-Di-Boc-1H-pyrazole-|-carboxamidine)

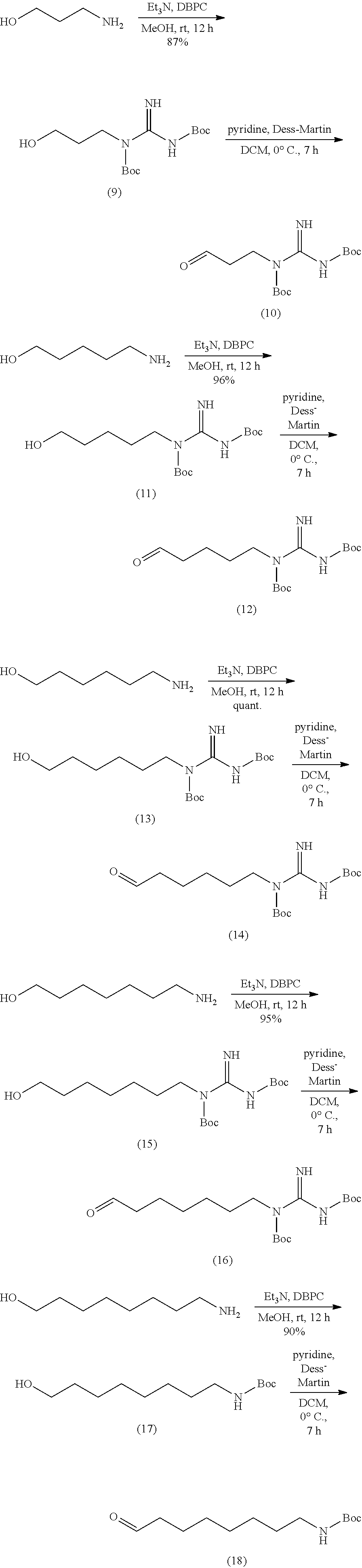

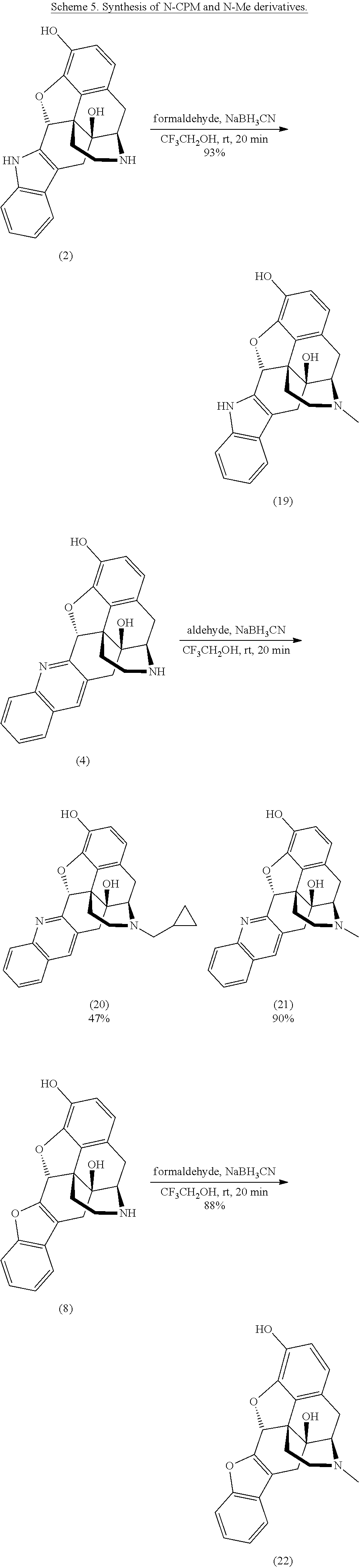

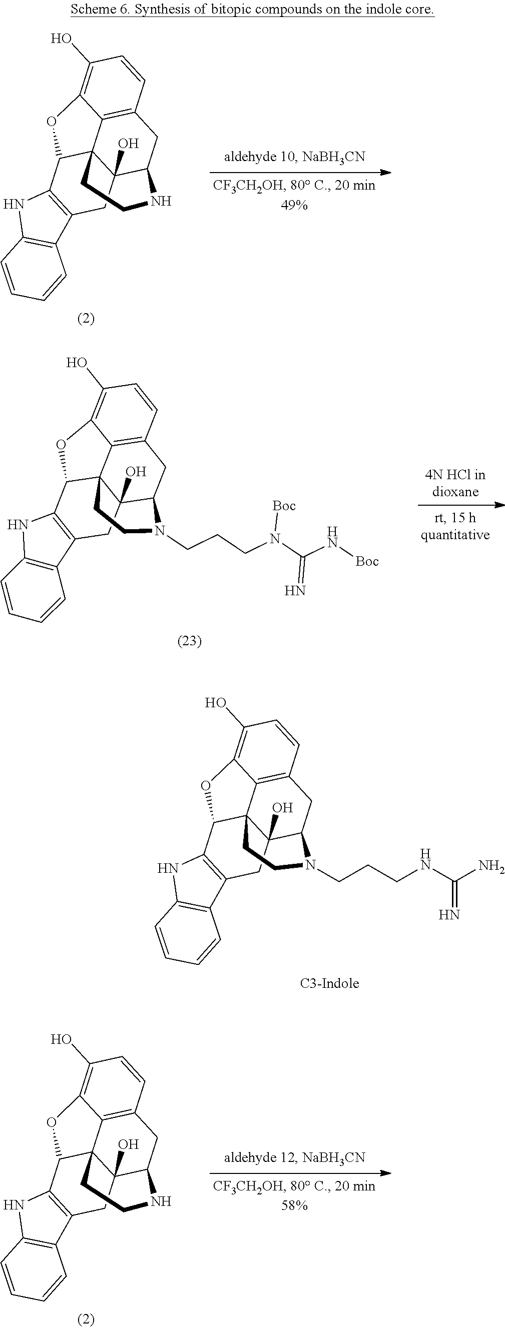

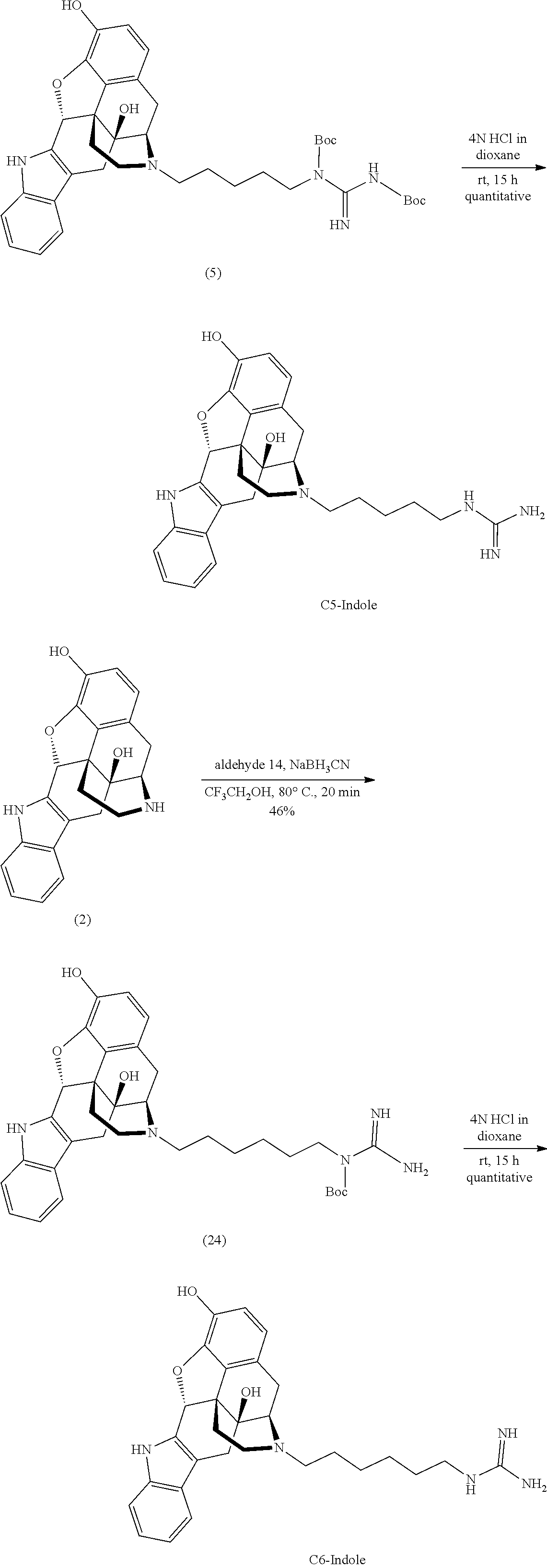

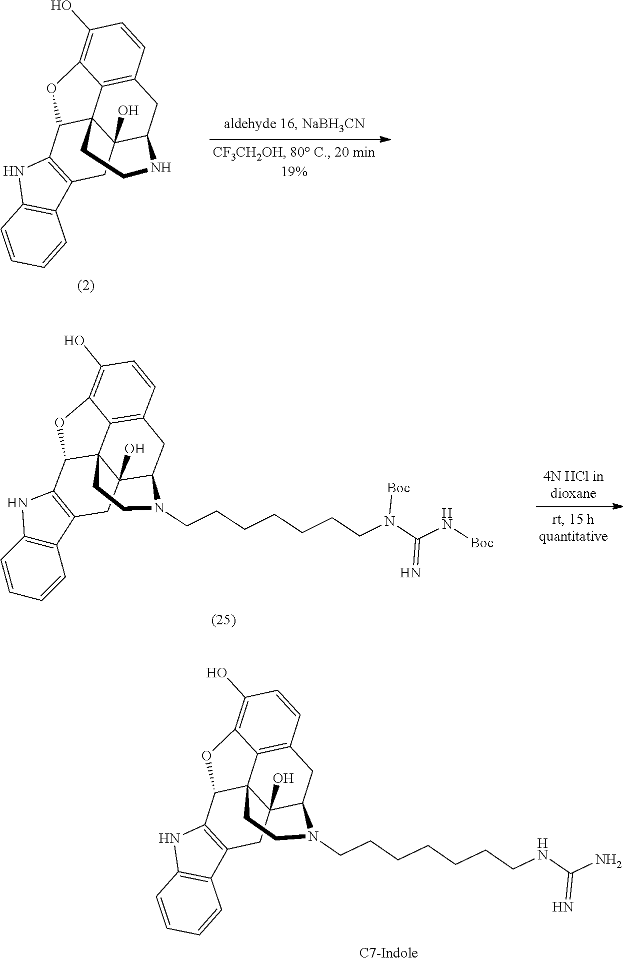

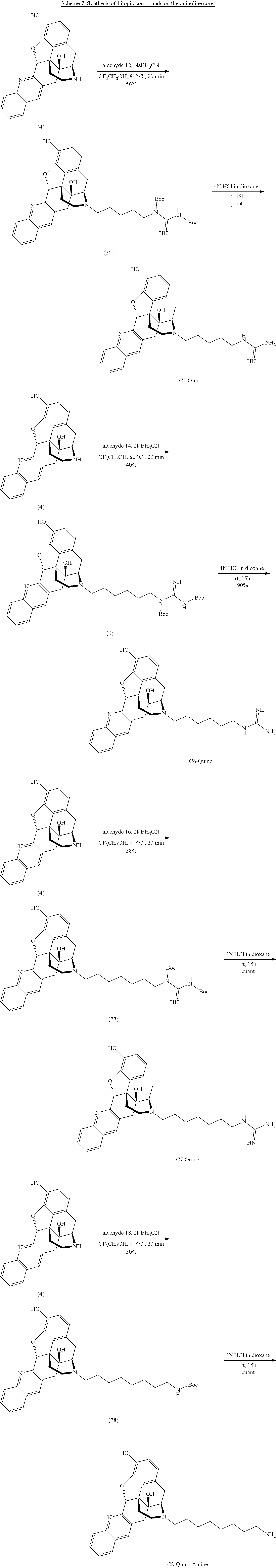

### Scheme 8. Synthesis of Bitopic Compounds on the Furane Core (Front Pharmacol. 2021 Nov. 3; 12:764885)

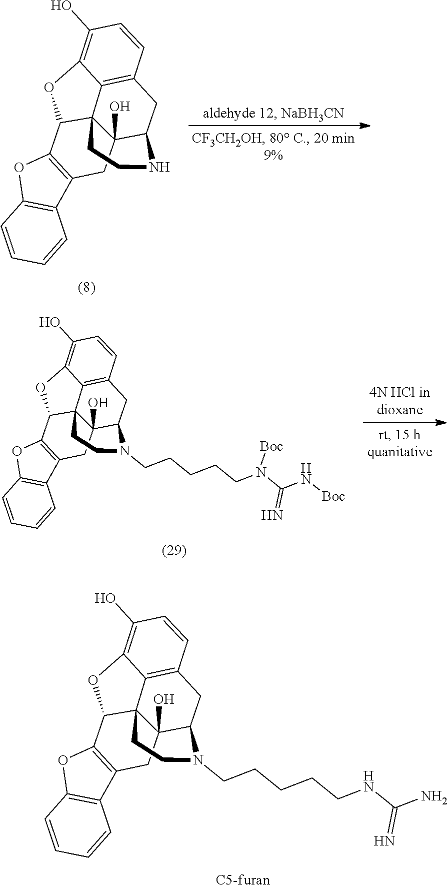

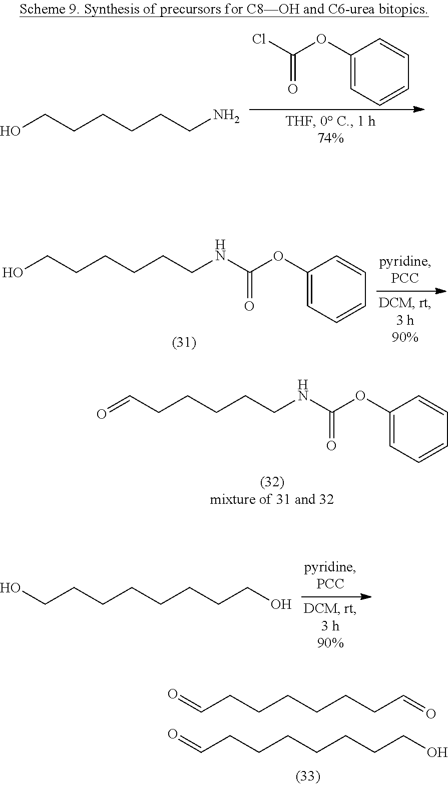

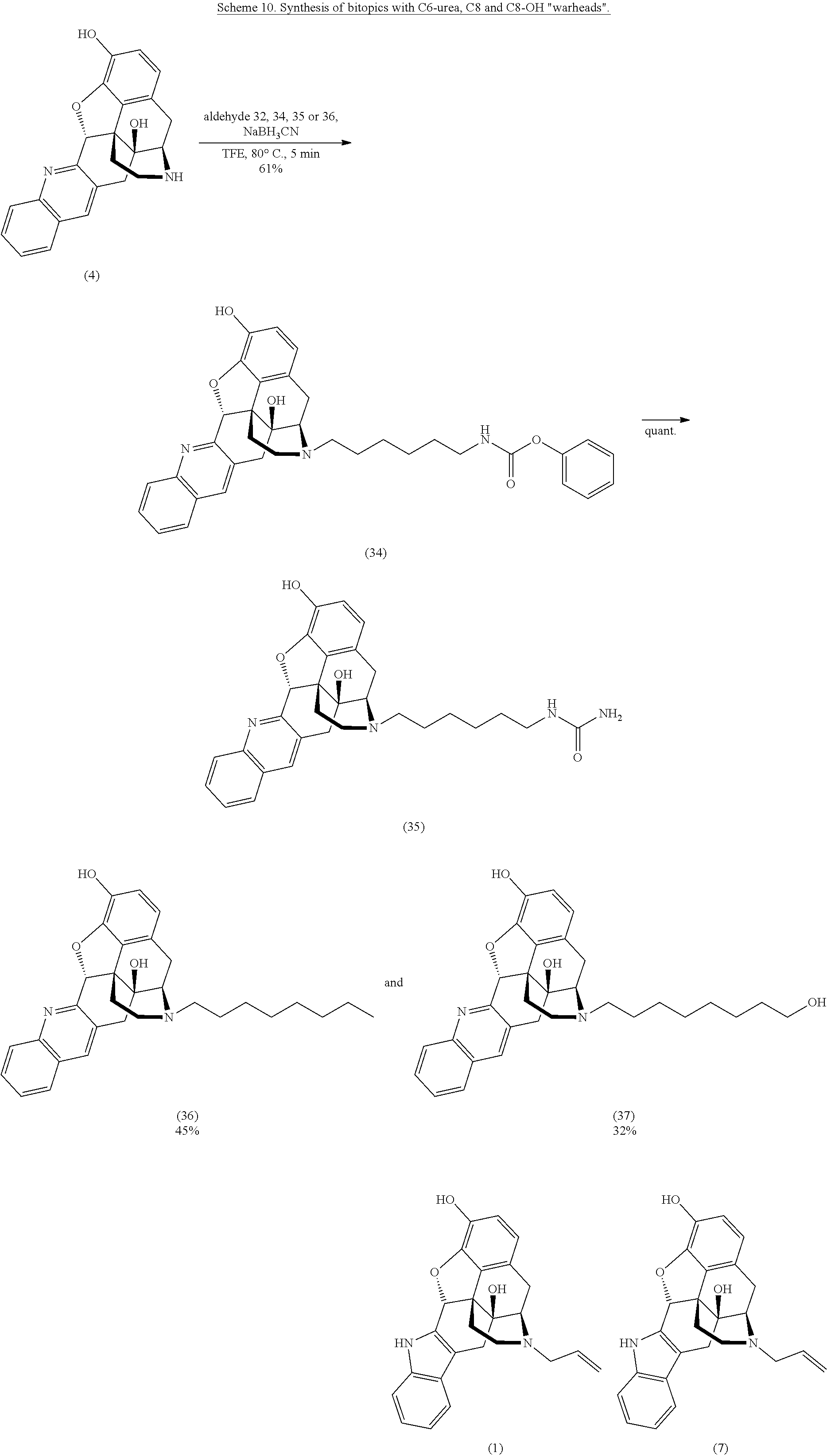

Nalindole (1) and naliben (7): For the synthesis of indole core a modified Fisher synthesis was employed. Naloxone·HCl (363.84, 1.0 mmol, 1.0 equiv) and phenylhydrazine (120 mg, 110 μl, 1.1 mmol, 1.1 equiv) or O-phenylhydroxylamine (290 mg, 2.0 mmol, 2.0 equiv.) were dissolved in trifluoroethanol (5 ml) and one drop of 10N HCl was added (presence of more water adversely affects the reaction). The reaction mixture was heated in microwave at 120° C. for 1 minute for 10 phenylhydrazine and for 30 minutes for O-phenylhydroxylamine, or, if necessary, until completion of the reaction. The mixture was quenched with sat. NaHCO₃ and extracted with DCM. Purification on silica using 0.5% to 6% MeOH in DCM gave the title compound as light yellow solid (95%).

(1): ¹H NMR (399 MHz, cd₃od) δ 7.39-7.29 (m, 2H), 7.20-7.02 (m, 1H), 6.96 (ddd, J=8.0, 7.0, 1.0 Hz, 1H), 6.66 (s, 1H), 6.03-5.88 (m, 1H), 5.73-5.58 (m, 3H), 3.99-3.89 (m, 1H), 3.80 (dd, J=15.3, 5.9 Hz, 2H), 3.44 (s, 1H), 3.28-3.16 (m, 2H), 2.92 (s, 1H), 2.92 (d, J=16.2 Hz, 1H), 2.77-2.60 (m, 2H), 1.92 (d, J=13.7 Hz, 1H). ¹³C NMR (100 MHz, cd₃od) δ 144.75, 141.89, 138.73, 130.09, 130.00, 127.73, 127.63, 126.47, 123.47, 122.35, 120.30, 119.84, 119.28, 119.24, 112.18, 109.22, 85.01, 73.49, 63.89, 56.61, 47.80, 47.28, 29.97, 29.62, 24.37.

(7): ¹H NMR (399 MHZ, cd₃od) δ 7.46 (t, J=7.3 Hz, 2H), 7.40-7.26 (m, 1H), 7.20 (t, J=7.5 Hz, 1H), 6.73-6.63 (m, 2H), 5.96 (dq, J=16.2, 9.0 Hz, 1H), 5.73-5.61 (m, 3H), 3.98 (t, J=11.2 Hz, 1H), 3.85 (t, J=11.4 Hz, 2H), 3.48 (s, 1H), 3.29-3.20 (m, 1H), 3.01-2.86 (m, 2H), 2.80-2.61 (m, 2H), 1.99 (d, J=13.8 Hz, 1H). ¹³C NMR (100 MHz, cd₃od) δ 176.03, 171.09, 156.80, 154.52, 148.80, 144.52, 142.11, 129.49, 128.39, 126.33, 123.77, 120.72, 120.47, 119.57, 115.30, 112.22, 83.85, 73.30, 63.52, 56.69, 49.42, 49.20, 48.99, 48.78, 48.56, 48.35, 48.14, 47.07, 29.93, 29.48, 24.22.

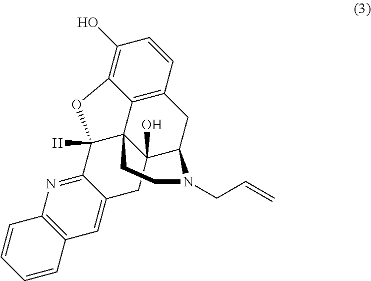

Allylquino (3): Naloxone·HCl (728 mg, 2.0 mmol, 1.0 equiv), 2-aminobenzaldehyde (480 mg, 4.0 mmol, 2.0 equiv) and p-TsOH (340 mg, 2.0 mmol, 1.0 equiv) were mixed in trifluoroethanol and refluxed for 14 h. After cooling the mixture was suspended in DCM, and the product was extracted into 1N NaOH. If an emulsion forms, that can be broken by the addition of a few drops of MeOH. The water phase was washed with DCM, which was checked for the presence of product. To the water phase 1N HCl was added until pH 8-9 was reached and the product was extracted into DCM. After drying over Na₂SO₄ and removal of the solvent, the product is usually sufficiently pure for deallylation, but also can be further purified on silica using 0.5% to 6% MeOH in DCM (78%).

¹H NMR (399 MHz, cdcl₃) δ 7.83 (dd, J=7.9, 1.8 Hz, 1H), 7.61 (s, 1H), 7.52 (dd, J=7.8, 1.8 Hz, 1H), 7.32 (pd, J=6.9, 1.7 Hz, 2H), 6.72 (d, J=8.1 Hz, 1H), 6.57 (d, J=8.1 Hz, 1H), 5.79 (ddt, J=16.6, 10.0, 6.4 Hz, 1H), 5.60 (s, 1H), 5.20 (dd, J=17.2, 1.7 Hz, 1H), 5.15 (dd, J=10.1, 1.7 Hz, 1H), 3.22-3.11 (m, 2H), 3.14-3.10 (m, 1H), 3.08 (d, J=6.3 Hz, 1H), 2.75 (dd, J=15.9, 1.6 Hz, 1H), 2.69 (d, J=3.8 Hz, 1H), 2.64 (dd, J=12.6, 6.1 Hz, 1H), 2.56 (q, J=4.1 Hz, 1H), 2.36-2.20 (m, 1H), 1.78-1.66 (m, 1H). ¹³C NMR (100 MHz, cdcl₃) δ 154.09, 146.80, 144.24, 139.28, 136.50, 135.32, 130.51, 128.76, 128.61, 128.08, 127.78, 126.83, 126.69, 124.47, 119.20, 118.06, 117.86, 90.43, 71.64, 61.79, 57.88, 47.18, 43.30, 36.16, 31.86, 23.15.

Deallylation. 1,3-dimethylbarbituric acid (286 mg, 1.83 mmol) freshly purified (suspended in cold methanol, filtered and dried under vacuum) catalyst Pd(PPh₃)₄ (176 mg, 0.15 mmol) were added to a flame dried vial containing the corresponding allyl derivative (3.05 mmol) and a magnetic stirring bar. The vial was sealed, flushed with argon, 30 mL anhydrous DCM was added, and the yellow reaction mixture was heated to reflux at 40° C. overnight. If the amount of precipitate withing the first few hours precluded stirring, more DCM was added, and the mixture was sonicated briefly. The reaction mixture was cooled to room temperature, 2 equivalents of 4N HCl in dioxane was added and the mixture was filtered. The residue was washed several times with DCM and dried. The products were usually sufficiently pure for further reactions, but further purification can be achieved on a short silica pad, eluting the nor compounds using 20% MeOH (containing 0.35N NH₃) in DCM.

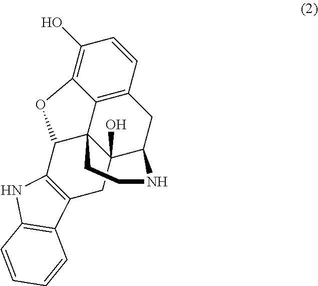

(2): ¹H NMR (400 MHZ, cd₃od) δ 7.34 (dd, J=16.1, 8.0 Hz, 2H), 7.08 (t, J=7.8 Hz, 1H), 6.94 (t, J=7.7 Hz, 1H), 6.63 (d, J=2.5 Hz, 2H), 5.67 (s, 1H), 3.87 (d, J=6.5 Hz, 1H), 3.50-3.39 (m, 1H), 3.30 (dd, J=16.2, 3.0 Hz, 5H), 3.25-3.13 (m, 2H), 3.02-2.88 (m, 2H), 2.72-2.58 (m, 2H), 1.87 (d, J=13.5 Hz, 1H). ¹³C NMR (101 MHz, cd₃od) δ 144.79, 141.76, 138.62, 130.23, 130.00, 127.65, 123.47, 122.71, 120.22, 119.86, 119.34, 119.18, 112.28, 109.40, 85.30, 72.35, 59.16, 47.90, 37.97, 29.73, 29.69, 28.45.

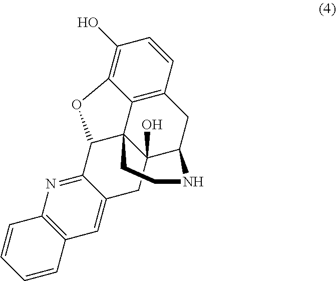

(4): ¹H NMR (399 MHz, cd₃od) δ 7.81-7.74 (m, 1H), 7.70 (s, 1H), 7.59 (dd, J=8.3, 1.4 Hz, 1H), 7.46 (ddd, J=8.4, 6.9, 1.4 Hz, 1H), 7.34 (ddd, J=8.1, 6.8, 1.2 Hz, 1H), 6.78-6.63 (m, 2H), 5.56 (s, 1H), 3.95 (d, J=6.1 Hz, 1H), 3.36 (d, J=12.9 Hz, 1H), 3.33 (s, 3H), 3.30-3.20 (m, 1H), 3.01 (td, J=13.3, 4.2 Hz, 1H), 2.89 (d, J=16.6 Hz, 1H), 2.76-2.62 (m, 2H), 1.84 (dd, J=13.5, 3.9 Hz, 1H). ¹³C NMR (100 MHz, cd₃od) δ 154.38, 147.68, 145.20, 141.58, 138.89, 130.66, 130.05, 128.96, 128.38, 128.24, 128.16, 127.68, 122.86, 121.09, 119.60, 90.39, 70.94, 58.48, 47.13, 37.84, 36.55, 29.62, 28.89.

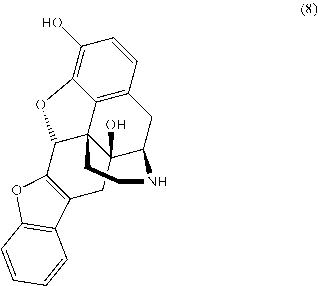

(8): ¹H NMR (399 MHz, cd₃od) δ 7.44 (t, J=7.1 Hz, 2H), 7.28 (t, J=7.7 Hz, 1H), 7.18 (t, J=7.5 Hz, 1H), 6.65 (s, 2H), 5.62 (s, 1H), 3.87 (d, J=6.5 Hz, 1H), 3.54-3.39 (m, 1H), 3.25-3.13 (m, 2H), 2.97 (dd, J=14.2, 10.4 Hz, 1H), 2.87 (d, J=16.5 Hz, 1H), 2.73-2.58 (m, 2H), 1.96-1.88 (m, 1H). ¹³C NMR (100 MHz, cd₃od) δ 156.73, 148.77, 144.61, 142.09, 129.70, 128.45, 126.27, 123.72, 122.57, 120.57, 120.44, 119.49, 115.34, 112.18, 84.08, 72.04, 58.81, 40.15, 37.76, 29.62, 29.50, 28.33.

Preparation of di-BOC-protected guano alcohols. The corresponding amino-alcohol (10 mmol, 1.0 equiv), pyrazol (3.4 g, 11 mmol, 1.1 equiv) and triethyl amine (2.0 g, 20 mmol, 2.0 equiv) were mixed in MeOH (50 mL) and stirred overnight. MeOH was removed under vacuum, the residue was redissolved in DCM and washed 3 times with 0.01N HCl or until the water phase reached pH 3. Finally, the organic phase was washed with diluted K₂CO₃ solution, dried over Na₂SO₄ and the solvent was removed on rotary evaporator. The product was used without further purification in the next step, analytical samples were obtained by purification using 20% to 50% EtOAc in hexane on silica.

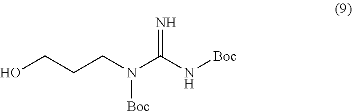 [SMILES: CC(C)(C)OC(=O)NC(=N)N(CCCO)C(=O)OC(C)(C)C]

(9): ¹H NMR (399 MHZ, cdcl₃ with a few drops of cd₃od) δ 3.62 (t, J=5.7 Hz, 2H), 3.52 (t, J=6.4 Hz, 2H), 1.81-1.70 (m, 2H), 1.52 (s, 9H), 1.49 (s, 9H). ¹³C NMR (100 MHz, cdcl₃) δ 167.91, 161.80, 157.98, 88.61, 84.67, 63.35, 42.27, 37.18, 33.07, 32.89.

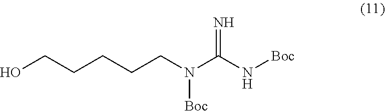 [SMILES: CC(C)(C)OC(=O)NC(=N)N(CCCCCO)C(=O)OC(C)(C)C]

(11): ¹H NMR (399 MHz, cdcl₃ with a few drops of cd₃od) δ 3.59 (t, J=6.6 Hz, 2H), 3.39 (d, J=7.1 Hz, 2H), 1.69-1.56 (m, 4H), 1.51 (d, J=5.8 Hz, 18H), 1.43 (tdd, J=9.2, 6.2, 3.4 Hz, 2H). ¹³C NMR (100 MHz, cdcl₃) δ 168.37, 161.15, 158.17, 88.44, 84.57, 66.89, 45.78, 37.08, 33.82, 33.16, 32.92, 28.08.

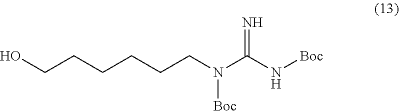 [SMILES: CC(C)(C)OC(=O)NC(=N)N(CCCCCCO)C(=O)OC(C)(C)C]

(13): ¹H NMR (399 MHz, cdcl₃ with a few drops of cd₃od) δ 3.58 (t, J=6.6 Hz, 2H), 3.38 (t, J=7.2 Hz, 2H), 1.59 (dt, J=13.0, 6.6 Hz, 2H), 1.51 (s, 9H), 1.50 (s, 9H), 1.49 (s, 1H), 1.46-1.34 (m, 5H). ¹³C NMR (100 MHz, cdcl₃) δ 163.14, 155.87, 152.94, 83.19, 79.32, 61.86, 40.52, 32.10, 28.69, 27.93, 27.69, 26.41, 25.18.

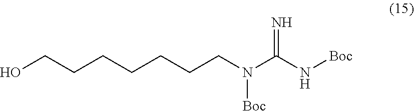 [SMILES: CC(C)(C)OC(=O)NC(=N)N(CCCCCCCO)C(=O)OC(C)(C)C]

(15): ¹H NMR (399 MHz, cdcl₃) δ 11.47 (s, 1H), 8.26 (t, J=5.1 Hz, 1H), 3.60 (t, J=6.6 Hz, 2H), 3.37 (td, J=7.2, 5.0 Hz, 2H), 1.58-1.50 (m, 4H), 1.46 (d, J=3.7 Hz, 18H), 1.39-1.29 (m, 2H), 1.32 (s, 5H). ¹³C NMR (100 MHz, cdcl₃) δ 164.83, 157.29, 154.52, 84.21, 80.42, 64.05, 42.07, 33.81, 30.16, 30.04, 29.49, 29.26, 27.94, 26.73.

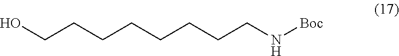 [SMILES: CC(C)(C)OC(=O)NCCCCCCCCO]

(17): ¹H NMR (399 MHz, cdcl₃) δ 3.52 (q, J=5.5 Hz, 2H), 3.01 (q, J=6.8 Hz, 2H), 1.47 (p, J=6.9 Hz, 2H), 1.39 (d, J=7.2 Hz, 2H), 1.35 (s, 9H), 1.22 (d, J=3.4 Hz, 8H). ¹³C NMR (100 MHz, cdcl₃) δ 157.25, 80.13, 63.82, 41.72, 33.83, 31.15, 30.48, 30.38, 29.57, 27.86, 26.84.

Preparation of di-BOC protected guano aldehydes. The corresponding guano alcohol (2.0 mmol, 1.0 equiv) and triethyl amine (7.5 mmol, 3.75 equiv) were dissolved in DCM (20 mL) and cooled in an ice bath. Dess-Martin periodinate (2.5 mmol, 1.25 equiv) was dissolved in DCM (20 mL) and added slowly. The mixture was allowed to warm to room temperature and was stirred for 3 hours or until the completion of the reaction. Saturated K₂CO₃ solution (3 mL) was added followed by powdered K₂CO₃ (3 g) in a few minutes. The resulting mixture was stirred for 10 minutes, the solids removed by filtration and washed with DCM. The organic phase was dried over Na₂SO₄ and the solvent was removed on rotary evaporator. The product was purified using 20% to 50% EtOAc in hexane on silica until at least 80% purity (by LC-MS) and used as such in the next step.

Reductive amination. The corresponding nor compounds (0.1-0.2 mmol, 1 equiv.) and aldehydes (0.5-1.0 mmol, 5 equiv.) were dissolved in trifluoroethanol (2-3 mL) and stirred at 75° C. for 3 minutes. NaBH₃CN (0.1-0.2 mmol, 1 equiv.) was added and the mixtures were heated at 75° C. for 2 more minutes. A 4 gram Redisep silica column was equilibrated with hexanes and the reaction mixtures were loaded directly after cooling and eluted first using a gradient of 0% to 100% EtOAc in hexanes and then 0% to 20% MeOH in DCM. The desired compounds usually eluted in the MeOH/DCM phase. The fractions containing the product were combined and repurified on a 4 gram silica column using 1% to 5% MeOH in DCM as eluent.

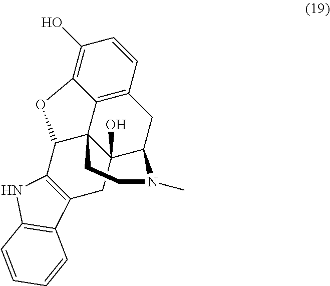

(19): ¹H NMR (399 MHz, cd₃od) δ 7.40-7.29 (m, 2H), 7.10 (ddd, J=8.3, 7.0, 1.2 Hz, 1H), 6.96 (ddd, J=8.0, 6.9, 1.1 Hz, 1H), 6.65 (s, 2H), 5.69 (s, 1H), 4.59 (s, 1H), 3.77 (d, J=6.6 Hz, 1H), 3.52-3.42 (m, 1H), 3.36-3.29 (m, 1H), 3.18 (dd, J=13.1, 4.6 Hz, 1H), 2.98-2.87 (m, 5H), 2.75-2.62 (m, 2H), 1.96-1.87 (m, 1H). ¹³C NMR (100 MHz, cd₃od) δ 144.76, 141.88, 138.74, 130.05, 130.00, 127.64, 123.47, 122.45, 120.25, 119.85, 119.25, 112.18, 109.21, 85.03, 73.61, 68.26, 47.20, 41.53, 29.90, 29.65, 24.88.

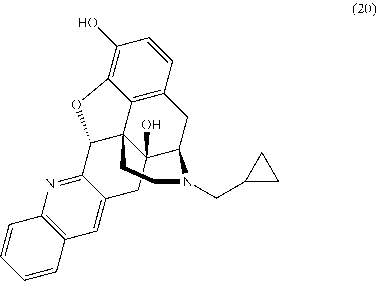

(20): ¹H NMR (399 MHz, cdcl₃) δ 8.49 (d, J=8.5 Hz, 1H), 8.38 (s, 1H), 8.22 (dd, J=8.3, 1.4 Hz, 1H), 8.15 (ddd, J=8.4, 6.9, 1.5 Hz, 1H), 7.99 (ddd, J=8.1, 6.8, 1.2 Hz, 1H), 7.16-7.02 (m, 2H), 6.05 (s, 1H), 5.80 (s, 0H), 3.97 (d, J=6.5 Hz, 1H), 3.64 (d, J=9.7 Hz, 1H), 3.41-3.21 (m, 3H), 3.10-2.87 (m, 3H), 2.40-2.28 (m, 1H), 2.06-1.94 (m, 1H), 1.75 (t, J=7.3 Hz, 1H), 1.47-1.33 (m, 2H), 1.33-1.24 (m, 1H), 1.15-1.02 (m, 2H), 0.75-0.64 (m, 2H). ¹³C NMR (100 MHZ, cdcl₃) δ 154.45, 146.92, 143.73, 139.64, 137.62, 130.12, 129.92, 128.46, 128.32, 127.87, 127.50, 127.40, 123.68, 119.79, 117.98, 89.87, 71.71, 59.50, 47.25, 44.15, 36.30, 31.76, 23.38, 13.36, 8.30, 4.43. HRMS calcd for C₂₇H₂₆N₂O₃ (MH+) 427.201619; found 427.201305.

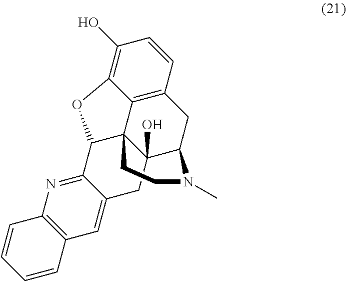

(21): ¹H NMR (399 MHz, cd₃od) δ 7.96 (d, J=8.6 Hz, 1H), 7.91 (s, 1H), 7.76 (d, J=8.2 Hz, 1H), 7.69-7.60 (m, 1H), 7.50 (t, J=7.5 Hz, 1H), 6.69-6.58 (m, 2H), 5.55 (s, 1H), 3.45-3.33 (m, 2H), 3.29 (p, J=1.7 Hz, 2H), 2.96-2.84 (m, 2H), 2.82 (d, J=4.4 Hz, 2H), 2.66 (s, 3H), 2.64-2.49 (m, 2H), 1.86-1.77 (m, 1H). ¹³C NMR (100 MHz, cd₃od) δ 155.11, 147.84, 144.89, 140.98, 138.61, 130.73, 130.63, 129.21, 128.80, 128.55, 128.27, 128.21, 124.31, 120.66, 118.98, 90.62, 72.61, 66.22, 47.11, 46.95, 42.57, 36.96, 31.58, 23.95. HRMS calcd for C₂₄H₂₂N₂O₃ (MH+) 387.170319; found 387.170058.

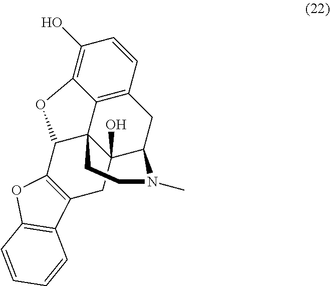

(22): ¹H NMR (399 MHz, cd₃od) δ 7.48 (ddd, J=7.1, 5.9, 1.2 Hz, 2H), 7.33 (tt, J=8.6, 1.3 Hz, 1H), 7.23 (td, J=7.5, 0.9 Hz, 1H), 6.72 (d, J=1.4 Hz, 2H), 5.70 (s, 1H), 3.86 (d, J=6.6 Hz, 1H), 3.51 (s, 1H), 3.37 (d, J=6.9 Hz, 1H), 3.25 (dd, J=12.8, 4.8 Hz, 1H), 3.01-2.91 (m, 5H), 2.81-2.64 (m, 2H), 1.99 (d, J=13.7 Hz, 1H). ¹³C NMR (100 MHz, cd₃od) δ 156.80, 148.80, 144.53, 142.11, 129.42, 128.39, 126.34, 123.78, 122.31, 120.73, 120.49, 119.61, 115.28, 112.22, 83.85, 73.39, 67.83, 41.57, 29.85, 29.46, 24.75.

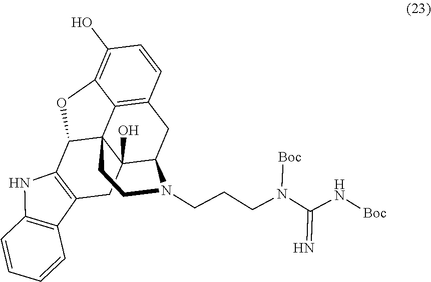

(23): ¹H NMR (399 MHz, cdcl₃) δ 11.48 (s, 1H), 8.46 (s, 1H), 8.37 (t, J=5.5 Hz, 1H), 7.34 (d, J=7.9 Hz, 1H), 7.16 (d, J=8.1 Hz, 1H), 7.06 (t, J=7.6 Hz, 1H), 6.96 (t, J=7.5 Hz, 1H), 6.53 (d, J=8.1 Hz, 1H), 6.43 (d, J=8.1 Hz, 1H), 5.64 (s, 1H), 3.44 (q, J=6.7 Hz, 2H), 3.12-3.03 (m, 2H), 2.83 (d, J=15.7 Hz, 1H), 2.79-2.69 (m, 1H), 2.58 (t, J=14.8 Hz, 1H), 2.48 (t, J=7.1 Hz, 3H), 2.31 (d, J=13.4 Hz, 1H), 2.26-2.11 (m, 1H), 1.71 (h, J=8.2 Hz, 3H), 1.46 (d, J=1.7 Hz, 9H), 1.41 (d, J=1.6 Hz, 9H), 1.40 (s, 2H). ¹³C NMR (100 MHz, cdcl₃) δ 163.64, 156.34, 153.48, 142.91, 139.18, 137.28, 130.75, 128.88, 126.71, 125.12, 122.80, 119.33, 119.14, 119.04, 117.35, 111.53, 111.43, 85.56, 83.41, 79.53, 72.97, 63.25, 52.07, 48.03, 43.76, 39.03, 31.26, 28.90, 28.36, 28.20, 27.29, 23.58.

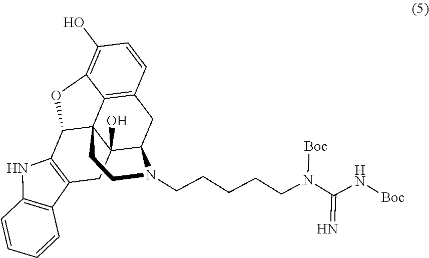

(5): ¹H NMR (400 MHZ, cdcl₃) δ 8.64 (t, J=10.9 Hz, 1H), 8.40-8.24 (m, 1H), 7.38 (d, J=7.8 Hz, 1H), 7.22 (d, J=8.2 Hz, 1H), 7.08 (t, J=7.6 Hz, 1H), 6.99 (d, J=7.6 Hz, 1H), 6.53 (d, J=8.0 Hz, 1H), 6.44 (d, J=8.2 Hz, 1H), 5.68 (s, 1H), 3.59-3.26 (m, 3H), 3.09 (d, J=5.2 Hz, 1H), 2.86 (d, J=15.8 Hz, 1H), 2.83-2.72 (m, 1H), 2.60 (d, J=15.8 Hz, 1H), 2.51 (d, J=10.9 Hz, 1H), 2.45 (d, J=7.2 Hz, 2H), 2.32 (d, J=12.1 Hz, 1H), 2.24 (d, J=12.1 Hz, 1H), 1.71 (d, J=12.1 Hz, 1H), 1.57 (q, J=8.0 Hz, 2H), 1.47 (s, 18H), 1.35 (s, 3H). ¹³C NMR (101 MHz, cdcl₃) δ 171.29, 163.65, 156.24, 153.41, 142.97, 139.30, 137.29, 130.76, 129.01, 126.71, 125.07, 122.67, 119.22, 118.99, 117.30, 111.40, 85.46, 83.19, 79.40, 72.89, 63.13, 60.49, 54.40, 48.06, 43.59, 40.97, 40.85, 31.45, 28.31, 28.12, 27.34, 24.59, 23.45, 21.11, 14.25.

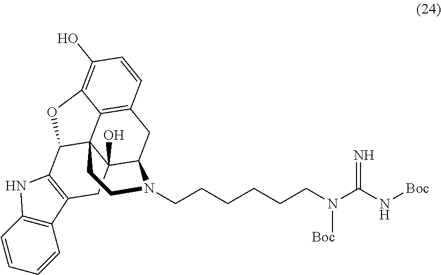

(24): ¹H NMR (500 MHZ, cdcl₃) δ 11.52 (s, 1H), 8.32 (d, J=5.8 Hz, 2H), 7.42 (d, J=7.9 Hz, 1H), 7.27 (d, J=8.2 Hz, 1H), 7.14 (ddd, J=8.2, 7.0, 1.2 Hz, 1H), 7.03 (ddd, J=8.0, 7.0, 1.0 Hz, 1H), 6.57 (d, J=8.1 Hz, 1H), 6.49 (d, J=8.2 Hz, 1H), 5.71 (s, 1H), 3.42 (td, J=7.3, 5.1 Hz, 2H), 3.20-3.11 (m, 2H), 2.90 (d, J=15.7 Hz, 1H), 2.86-2.78 (m, 1H), 2.64 (dd, J=15.7, 1.3 Hz, 1H), 2.59 (dd, J=11.2, 4.5 Hz, 1H), 2.51 (qd, J=7.4, 5.5 Hz, 2H), 2.39 (td, J=12.4, 4.8 Hz, 1H), 2.30 (td, J=11.9, 3.3 Hz, 1H), 1.77 (dt, J=11.4, 2.6 Hz, 1H), 1.65-1.51 (m, 4H), 1.51 (s, 9H), 1.50 (s, 9H), 1.39 (p, J=3.7 Hz, 4H). ¹³C NMR (100 MHz, cdcl₃) δ 164.82, 157.35, 154.55, 143.90, 140.17, 138.38, 131.80, 129.95, 127.81, 126.30, 123.92, 120.45, 120.28, 120.17, 118.34, 112.66, 112.50, 86.74, 84.30, 80.54, 73.98, 64.18, 55.62, 49.20, 42.08, 30.10, 29.96, 29.47, 29.28, 29.25, 28.65, 28.15, 27.89, 24.56.

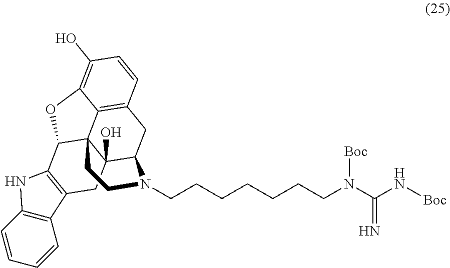

(25): ¹H NMR (399 MHz, cdcl₃) δ 11.47 (s, 1H), 8.36 (s, 1H), 8.27 (t, J=5.3 Hz, 1H), 7.36 (d, J=7.9 Hz, 1H), 7.20 (d, J=8.2 Hz, 1H), 7.07 (t, J=7.7 Hz, 1H), 6.96 (t, J=7.5 Hz, 1H), 6.49 (d, J=8.1 Hz, 1H), 6.41 (d, J=8.1 Hz, 1H), 5.68 (s, 1H), 5.26 (s, 1H), 3.35 (q, J=6.6 Hz, 2H), 3.15-3.06 (m, 2H), 2.86 (d, J=15.7 Hz, 1H), 2.81-2.70 (m, 1H), 2.59 (d, J=15.8 Hz, 1H), 2.52 (dd, J=11.6, 4.3 Hz, 1H), 2.45 (t, J=7.3 Hz, 2H), 2.33 (td, J=12.4, 4.6 Hz, 1H), 2.27-2.13 (m, 1H), 1.70 (d, J=12.0 Hz, 1H), 1.50 (dd, J=15.1, 7.9 Hz, 3H), 1.44 (d, J=2.9 Hz, 18H), 1.30 (s, 7H). ¹³C NMR (100 MHz, cdcl₃) δ 164.80, 157.30, 154.51, 143.84, 140.10, 138.35, 131.82, 129.93, 127.80, 126.40, 123.88, 120.42, 120.25, 118.28, 112.72, 86.75, 84.25, 80.49, 73.98, 64.21, 55.72, 49.24, 44.69, 42.12, 32.55, 30.32, 30.09, 29.93, 29.45, 29.25, 28.80, 28.38, 27.99, 24.52, 22.26.

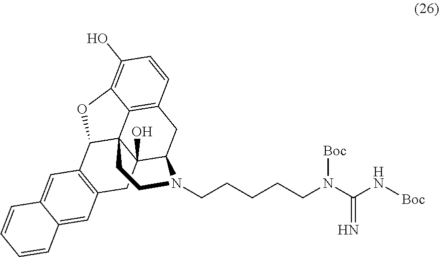

(26): ¹H NMR (399 MHz, cdcl₃) δ 11.46 (s, 1H), 8.29 (t, J=5.2 Hz, 1H), 7.95 (d, J=8.3 Hz, 1H), 7.75 (s, 1H), 7.61 (d, J=8.0 Hz, 1H), 7.45 (s, 1H), 7.38 (t, J=7.5 Hz, 1H), 6.68 (d, J=8.1 Hz, 1H), 6.56 (d, J=8.1 Hz, 1H), 5.61 (s, 1H), 3.46-3.31 (m, 3H), 3.04 (d, J=6.3 Hz, 1H), 2.82 (d, J=15.8 Hz, 1H), 2.74 (d, J=11.5 Hz, 1H), 2.73-2.64 (m, 1H), 2.57 (d, J=7.8 Hz, 1H), 2.48 (q, J=6.3 Hz, 2H), 2.34 (q, J=11.0 Hz, 2H), 1.80 (d, J=9.9 Hz, 1H), 1.60-1.54 (m, 2H), 1.45 (s, 12H), 1.42 (s, 9H), 1.37 (t, J=7.8 Hz, 2H). ¹³C NMR (100 MHz, cdcl₃) δ 164.81, 157.35, 155.32, 154.55, 148.40, 145.00, 140.04, 137.67, 131.60, 130.21, 130.14, 129.37, 129.14, 128.10, 125.91, 120.49, 118.51, 92.15, 84.29, 80.48, 72.74, 63.57, 55.49, 48.41, 44.79, 41.88, 37.42, 33.12, 30.90, 29.94, 29.50, 29.24, 28.27, 25.66, 24.61.

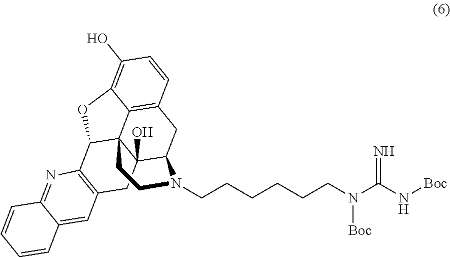

(6): ¹H NMR (500 MHz, cdcl₃) δ 11.51 (s, 1H), 8.31 (t, J=5.2 Hz, 1H), 8.00 (dq, J=8.5, 0.9 Hz, 1H), 7.76 (s, 1H), 7.68-7.62 (m, 1H), 7.56 (ddd, J=8.5, 6.8, 1.5 Hz, 1H), 7.44 (ddd, J=8.1, 6.9, 1.2 Hz, 1H), 6.69 (d, J=8.1 Hz, 1H), 6.60 (d, J=8.1 Hz, 1H), 5.67 (s, 1H), 3.41 (td, J=7.2, 5.1 Hz, 2H), 3.08 (d, J=6.5 Hz, 1H), 2.85 (dd, J=15.8, 1.7 Hz, 1H), 2.79-2.68 (m, 2H), 2.66-2.55 (m, 1H), 2.51 (tt, J=8.8, 4.3 Hz, 2H), 2.44-2.32 (m, 2H), 1.88-1.79 (m, 1H), 1.63-1.51 (m, 3H), 1.50 (s, 9H), 1.49 (s, 9H), 1.38 (p, J=3.5 Hz, 4H). ¹³C NMR (100 MHz, cdcl₃) δ 164.80, 157.30, 155.37, 154.52, 148.26, 145.06, 140.13, 137.64, 131.64, 130.12, 130.02, 129.37, 129.07, 128.03, 125.87, 120.42, 118.59, 91.93, 84.24, 80.45, 72.73, 63.53, 55.59, 48.45, 44.67, 42.04, 37.41, 33.21, 30.11, 29.48, 29.26, 29.21, 28.66, 28.14, 27.89, 24.49.

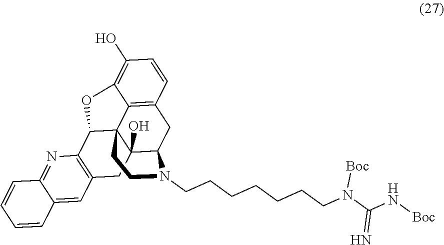

(27): ¹H NMR (399 MHz, cdcl₃) δ 11.44 (s, 1H), 8.24 (t, J=5.2 Hz, 1H), 7.94 (d, J=8.5 Hz, 1H), 7.71 (s, 1H), 7.58 (d, J=8.2 Hz, 1H), 7.46 (t, J=8.0 Hz, 1H), 7.36 (t, J=7.5 Hz, 1H), 6.62 (d, J=8.1 Hz, 1H), 6.52 (d, J=8.1 Hz, 1H), 5.54 (s, 1H), 3.96 (hept, J=6.1 Hz, 2H), 3.34 (q, J=6.6 Hz, 2H), 3.01 (d, J=6.4 Hz, 1H), 2.78 (d, J=15.9 Hz, 1H), 2.75-2.60 (m, 2H), 2.60-2.51 (m, 1H), 2.44 (td, J=7.0, 2.8 Hz, 1H), 2.29 (dt, J=8.1, 4.8 Hz, 2H), 1.50 (t, J=7.2 Hz, 2H), 1.42 (d, J=6.9 Hz, 19H), 1.28 (s, 7H), 1.12 (dd, J=8.4, 6.2 Hz, 13H). ¹³C NMR (100 MHz, cdcl₃) δ 164.77, 157.27, 155.56, 154.47, 148.13, 144.93, 140.55, 137.75, 131.49, 130.19, 129.64, 129.43, 129.09, 128.08, 128.02, 125.29, 120.46, 118.86, 91.35, 84.20, 80.41, 78.61, 78.29, 77.97, 72.70, 65.38, 63.56, 55.64, 48.33, 44.61, 42.06, 37.33, 33.15, 30.27, 30.06, 29.45, 29.21, 28.69, 28.32, 27.97, 26.36, 24.43.

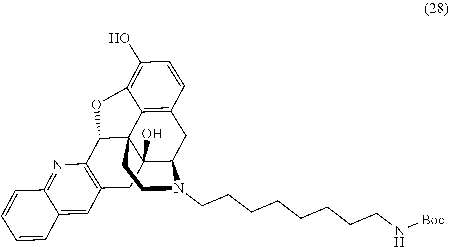

(28): ¹H NMR (399 MHz, cdcl₃) δ 7.98 (d, J=8.5 Hz, 1H), 7.76 (s, 1H), 7.63 (d, J=8.1 Hz, 1H), 7.52 (ddd, J=8.5, 6.8, 1.5 Hz, 1H), 7.41 (t, J=7.5 Hz, 1H), 6.65 (d, J=8.1 Hz, 1H), 6.55 (d, J=8.1 Hz, 1H), 5.62 (s, 1H), 4.88 (s, 1H), 4.50 (s, 2H), 4.08 (q, J=7.1 Hz, 1H), 3.21 (s, 0H), 3.16 (s, 1H), 3.06 (dd, J=11.9, 6.2 Hz, 4H), 2.82 (d, J=15.8 Hz, 1H), 2.77-2.63 (m, 2H), 2.63-2.55 (m, 1H), 2.47 (td, J=7.0, 2.9 Hz, 2H), 2.40-2.25 (m, 2H), 2.00 (s, 1H), 1.50-1.39 (m, 6H), 1.39 (s, 14H), 1.33-1.18 (m, 13H). ¹³C NMR (100 MHz, cdcl₃) δ 157.19, 155.42, 148.30, 144.92, 140.13, 137.71, 131.61, 130.23, 129.99, 129.46, 129.14, 128.10, 128.09, 125.84, 120.47, 118.51, 91.93, 72.75, 63.58, 61.61, 55.68, 48.49, 44.61, 41.76, 37.43, 33.26, 31.21, 30.58, 30.43, 29.61, 28.77, 28.41, 27.92, 24.48, 22.26, 15.38.

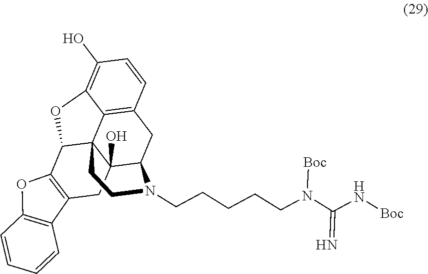

(29): ¹H NMR (399 MHz, cdcl₃) δ 11.50 (s, 2H), 8.33 (t, J=5.3 Hz, 2H), 7.40 (dd, J=17.6, 7.9 Hz, 4H), 7.26 (t, J=8.0 Hz, 1H), 7.17 (t, J=7.5 Hz, 2H), 6.70 (s, 2H), 5.61 (s, 2H), 4.88 (s, 3H), 3.40 (dt, J=8.5, 5.9 Hz, 4H), 3.15 (d, J=6.4 Hz, 2H), 3.09 (s, 1H), 3.04 (s, 1H), 2.83-2.71 (m, 3H), 2.69 (d, J=6.5 Hz, 1H), 2.64-2.53 (m, 3H), 2.57-2.46 (m, 5H), 2.36 (td, J=12.6, 4.9 Hz, 2H), 2.28-2.20 (m, 1H), 2.24-2.14 (m, 1H), 1.79 (s, 1H), 1.63-1.55 (m, 4H), 1.51 (dd, J=16.6, 9.2 Hz, 4H), 1.47 (s, 5H), 1.46 (s, 19H), 1.45 (s, 16H), 1.41 (d, J=8.0 Hz, 2H), 1.37 (d, J=8.4 Hz, 2H), 1.24 (d, J=1.7 Hz, 3H), 0.83 (s, 1H). ¹³C NMR (100 MHz, cdcl₃) δ 164.76, 157.38, 156.74, 154.56, 148.44, 143.21, 141.23, 132.55, 128.63, 126.36, 124.52, 124.01, 123.81, 120.99, 118.42, 117.37, 112.78, 86.10, 84.34, 80.54, 78.54, 78.22, 77.91, 73.53, 63.63, 55.46, 50.25, 44.27, 41.92, 32.45, 29.98, 29.92, 29.45, 29.26, 28.43, 25.68, 23.84.

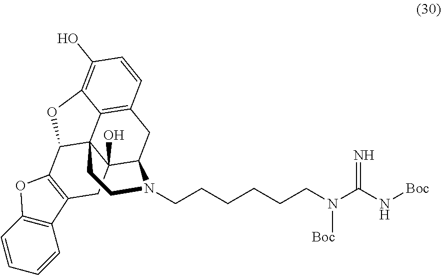

(30): ¹H NMR (399 MHz, cdcl₃) δ 11.50 (s, 1H), 8.30 (t, J=4.8 Hz, 1H), 7.43 (d, J=8.3 Hz, 1H), 7.37 (d, J=7.7 Hz, 1H), 7.29-7.21 (m, 1H), 7.15 (t, J=7.5 Hz, 1H), 6.64 (d, J=8.1 Hz, 1H), 6.56 (d, J=8.2 Hz, 1H), 5.62 (s, 1H), 3.40 (q, J=6.9 Hz, 2H), 3.21-3.13 (m, 1H), 3.11 (d, J=6.4 Hz, 1H), 2.81 (d, J=5.7 Hz, 1H), 2.77 (d, J=2.7 Hz, 1H), 2.64-2.53 (m, 2H), 2.50 (dp, J=12.0, 5.5 Hz, 2H), 2.42-2.23 (m, 2H), 1.84-1.76 (m, 1H), 1.61-1.49 (m, 2H), 1.48 (s, 8H), 1.37 (p, J=3.5 Hz, 4H), 1.24 (s, 1H). ¹³C NMR (100 MHz, cdcl₃) δ 164.85, 157.33, 156.73, 154.56, 148.82, 144.05, 140.19, 131.34, 129.49, 128.77, 126.22, 123.70, 120.92, 120.40, 118.25, 117.29, 112.79, 85.78, 84.26, 80.48, 73.79, 64.00, 55.62, 50.14, 44.52, 42.06, 32.57, 30.90, 30.14, 30.01, 29.50, 29.28, 28.18, 27.92, 24.51.

Boc deprotection. The resulting boc protected guanidines and amines (0.03-0.1 mmol) were dissolved in 4N HCl (2 mL) under argon and stirred for between 3 h to 12 h at room temperature. Diethyl ether (6 mL) was added, and the mixture was sonicated until a fine precipitate formed, the vials were centrifuged and the solvent decanted. This process was repeated three times, then the solids were dried at room temperature. Purity was checked by NMR and if necessary further purification was performed on a reverse phase chromatography using acetonitrile (0.05% TFA) and water (0.05% TFA) as eluent, 10% to 40% acetonitrile gradient.

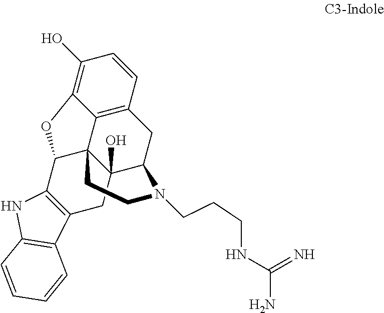

C3-Indole: ¹H NMR (399 MHz, cd₃od) δ 7.32 (dd, J=8.0, 2.8 Hz, 2H), 7.09 (t, J=7.6 Hz, 1H), 6.95 (t, J=7.5 Hz, 1H), 6.70-6.61 (m, 2H), 5.66 (s, 1H), 3.85 (d, J=6.2 Hz, 1H), 3.30 (dd, J=14.4, 7.7 Hz, 5H), 3.11 (t, J=12.7 Hz, 2H), 2.91 (d, J=16.0 Hz, 1H), 2.76 (d, J=13.0 Hz, 1H), 2.70-2.54 (m, 2H), 2.12 (dq, J=12.6, 6.2 Hz, 1H), 1.92 (dq, J=12.3, 6.2 Hz, 1H), 1.77 (d, J=13.4 Hz, 1H). ¹³C NMR (100 MHz, cd₃od) δ 163.01, 158.88, 158.83, 144.90, 142.03, 138.89, 130.25, 130.23, 127.82, 123.69, 122.50, 120.57, 120.07, 119.53, 119.47, 112.42, 109.43, 85.18, 73.59, 64.24, 51.93, 48.12, 47.82, 39.53, 30.18, 29.76, 24.96, 24.73. HRMS calcd for C₂₆H₂₉N₅O₃ (MH+) 460.234316; found 460.234129.

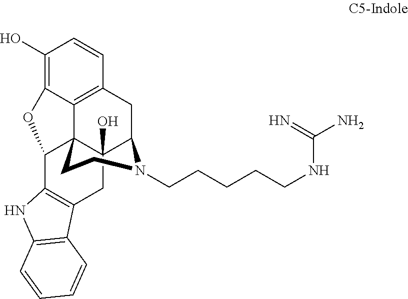

C5-Indole: ¹H NMR (399 MHz, cd₃od) δ 7.33 (dd, J=8.0, 5.8 Hz, 2H), 7.10 (t, J=7.6 Hz, 1H), 6.96 (t, J=7.5 Hz, 1H), 6.68 (s, 2H), 5.67 (s, 1H), 3.85 (d, J=5.7 Hz, 1H), 3.39-3.28 (m, 2H), 3.22 (h, J=6.3 Hz, 3H), 3.02 (qd, J=12.7, 4.5 Hz, 2H), 2.91 (d, J=16.0 Hz, 1H), 2.75 (td, J=12.7, 3.6 Hz, 1H), 2.70-2.54 (m, 2H), 1.90-1.68 (m, 2H), 1.66 (q, J=7.5 Hz, 3H), 1.45 (p, J=7.6 Hz, 2H). ¹³C NMR (100 MHz, cd₃od) δ 163.18, 162.83, 158.74, 158.69, 144.90, 142.01, 139.04, 138.89, 130.41, 130.31, 130.27, 127.87, 127.83, 123.69, 122.60, 120.61, 120.05, 119.54, 119.43, 116.70, 112.43, 109.45, 85.18, 73.59, 63.66, 54.22, 47.99, 47.88, 42.21, 42.08, 30.19, 29.76, 29.36, 24.96, 24.62. HRMS calcd for C₂₈H₃₃N₅O₃ (MH+) 488.265616; found 488.265851.

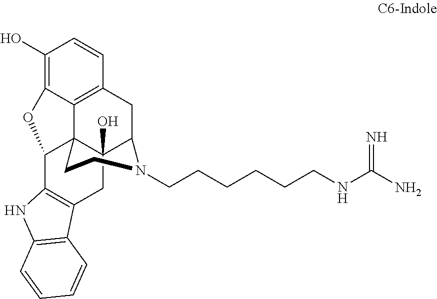

C6-Indole: ¹H NMR (399 MHz, cd₃od) δ 7.34 (dd, J=17.9, 8.1 Hz, 2H), 7.10 (t, J=7.6 Hz, 1H), 6.96 (t, J=7.5 Hz, 1H), 6.65 (s, 2H), 5.69 (s, 1H), 3.89 (d, J=6.4 Hz, 1H), 3.39 (s, 1H), 3.32 (d, J=6.7 Hz, 2H), 3.19 (t, J=7.1 Hz, 3H), 3.09 (d, J=12.3 Hz, 1H), 2.95 (d, J=15.9 Hz, 2H), 2.78-2.60 (m, 2H), 1.91 (d, J=15.2 Hz, 2H), 1.64 (s, 4H), 1.47 (s, 5H). ¹³C NMR (100 MHZ, cd₃od) δ 158.46, 144.76, 141.88, 138.74, 130.12, 130.02, 127.64, 123.47, 122.33, 120.25, 119.84, 119.24, 112.18, 109.22, 85.02, 73.39, 63.48, 54.21, 49.50, 49.42, 49.21, 48.99, 48.78, 48.57, 48.36, 48.14, 47.98, 47.77, 42.10, 30.07, 29.63, 29.50, 27.09, 27.04, 25.10, 24.46. HRMS calcd for C₂₉H₃₅N₅O₃ (MH+) 502.281266; found 502.281317.

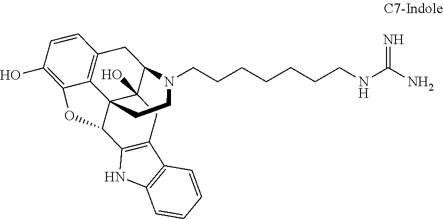

C7-Indole: ¹H NMR (399 MHz, cd₃od) δ 7.40-7.29 (m, 2H), 7.10 (t, J=7.6 Hz, 1H), 6.96 (t, J=7.5 Hz, 1H), 6.65 (s, 2H), 5.70 (s, 1H), 3.89 (d, J=6.5 Hz, 1H), 3.40 (s, 1H), 3.36-3.28 (m, 1H), 3.22-3.02 (m, 4H), 2.95 (s, 1H), 2.95 (d, J=16.4 Hz, 1H), 2.78-2.63 (m, 2H), 1.92 (d, J=13.9 Hz, 1H), 1.85 (s, 1H), 1.62 (t, J=7.1 Hz, 3H), 1.45 (s, 8H). ¹³C NMR (100 MHz, cd₃od) δ 171.13, 158.44, 144.76, 141.89, 138.74, 130.13, 130.01, 127.64, 123.47, 122.33, 120.24, 119.84, 119.23, 112.18, 109.22, 85.03, 73.39, 63.45, 54.28, 48.00, 47.78, 42.21, 30.08, 29.73, 29.64, 29.60, 27.39, 27.34, 25.19, 24.45. HRMS calcd for C₃₀H₃₇N₅O₃ (MH+) 516.296917; found 516.297086.

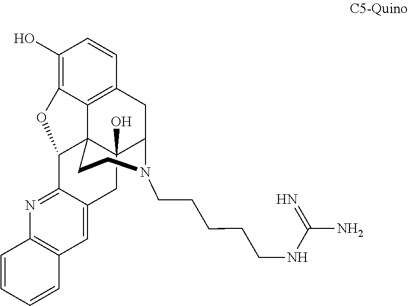

C5-Quino: ¹H NMR (399 MHZ, cd₃od) δ 8.10 (s, 1H), 7.97 (d, J=8.6 Hz, 1H), 7.79 (d, J=8.3 Hz, 1H), 7.70 (ddd, J=8.5, 6.8, 1.4 Hz, 1H), 7.54 (t, J=7.6 Hz, 1H), 6.78-6.68 (m, 2H), 5.72 (s, 1H), 3.98 (d, J=6.6 Hz, 1H), 3.48 (s, 1H), 3.39 (td, J=12.2, 5.1 Hz, 1H), 3.29-3.12 (m, 4H), 3.14-2.95 (m, 2H), 2.90-2.74 (m, 2H), 2.00-1.83 (m, 2H), 1.79-1.63 (m, 3H), 1.48 (p, J=7.7 Hz, 2H). ¹³C NMR (100 MHz, cd₃od) δ 158.52, 153.68, 145.90, 144.86, 141.70, 141.32, 132.15, 129.46, 129.30, 129.03, 128.58, 128.10, 126.75, 122.34, 121.43, 119.69, 88.80, 71.90, 62.75, 54.28, 47.78, 47.00, 41.89, 36.66, 29.91, 29.19, 24.82, 24.44, 24.40. HRMS calcd for C₂₉H₃₃N₅O₃ (MH+) 500.265616; found 500.265356.

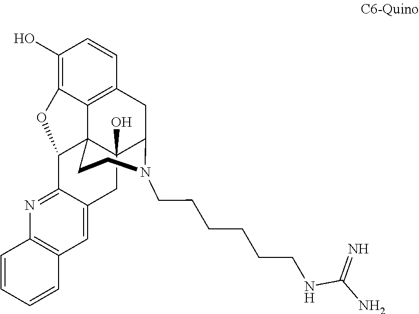

C6-Quino: ¹H NMR (399 MHz, cd₃od) δ 8.15 (s, 1H), 8.02 (d, J=8.6 Hz, 1H), 7.85 (dd, J=8.4, 1.4 Hz, 1H), 7.75 (ddd, J=8.5, 6.9, 1.4 Hz, 1H), 7.58 (ddd, J=8.2, 6.9, 1.1 Hz, 1H), 6.77-6.67 (m, 2H), 5.72 (s, 1H), 3.96 (d, J=6.6 Hz, 1H), 3.47 (s, 1H), 3.39 (td, J=12.0, 5.0 Hz, 1H), 3.28-2.96 (m, 7H), 2.94-2.86 (m, 1H), 2.80 (td, J=13.4, 4.9 Hz, 1H), 2.03-1.93 (m, 1H), 1.93-1.83 (m, 1H), 1.73-1.58 (m, 3H), 1.48 (dp, J=7.8, 4.2 Hz, 4H). ¹³C NMR (100 MHz, cd₃od) δ 158.68, 154.04, 146.43, 145.07, 141.90, 141.29, 132.26, 129.67, 129.57, 129.21, 128.78, 128.30, 127.23, 122.50, 121.55, 119.87, 89.15, 72.11, 62.95, 54.60, 48.01, 47.21, 42.28, 36.91, 30.12, 29.66, 27.25, 27.21, 25.29, 24.58. HRMS calcd for C₃₀H₃₅N₅O₃ (MH+) 514.281266; found 514.280867.

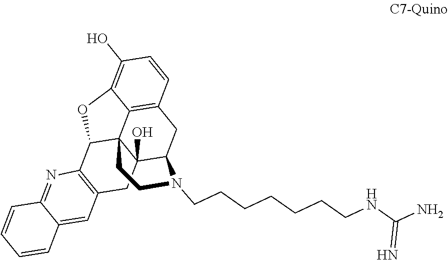

C7-Quino: ¹H NMR (399 MHz, cd₃od) δ 8.13 (s, 1H), 8.02 (d, J=8.6 Hz, 1H), 7.84 (d, J=8.2 Hz, 1H), 7.74 (ddd, J=8.5, 6.9, 1.4 Hz, 1H), 7.57 (t, J=7.6 Hz, 1H), 6.72 (q, J=8.2 Hz, 2H), 5.71 (s, 1H), 3.96 (d, J=6.6 Hz, 1H), 3.47 (s, 1H), 3.38 (td, J=12.0, 5.0 Hz, 1H), 3.28-3.17 (m, 2H), 3.17 (s, 1H), 3.17-2.96 (m, 3H), 2.94-2.74 (m, 2H), 1.97 (dd, J=13.7, 3.6 Hz, 1H), 1.89-1.83 (m, 1H), 1.69 (s, 1H), 1.60 (p, J=7.2 Hz, 2H), 1.44 (s, 4H), 1.44 (t, J=10.1 Hz, 2H). ¹³C NMR (100 MHz, cd₃od) δ 158.47, 153.91, 146.39, 144.89, 141.69, 140.88, 131.94, 129.49, 129.35, 128.94, 128.54, 128.06, 127.17, 122.30, 121.32, 119.65, 89.05, 71.92, 62.72, 54.47, 47.80, 47.01, 42.18, 36.73, 29.93, 29.66, 29.55, 27.32, 27.28, 25.16, 24.37. HRMS calcd for C₃₁H₃₇N₅O₃ (MH+) 528.296917; found 528.297076.

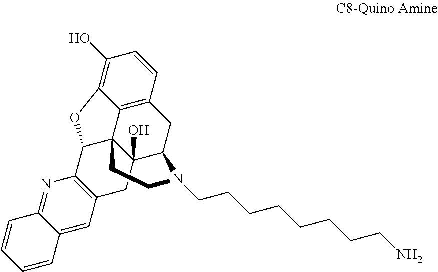

C8-Quino Amine: ¹H NMR (399 MHz, cd₃od) δ 8.16 (s, 1H), 8.06 (d, J=8.6 Hz, 1H), 7.87 (d, J=8.2 Hz, 1H), 7.78 (ddd, J=8.4, 6.9, 1.4 Hz, 1H), 7.61 (ddd, J=8.1, 6.9, 1.2 Hz, 1H), 6.77-6.65 (m, 2H), 5.70 (s, 1H), 3.95 (d, J=6.6 Hz, 1H), 3.47 (s, 1H), 3.39 (td, J=12.2, 5.0 Hz, 1H), 3.29-2.98 (m, 4H), 2.92 (dd, J=15.8, 7.7 Hz, 3H), 2.89-2.73 (m, 1H), 1.99 (d, J=17.5 Hz, 1H), 1.86 (s, 1H), 1.67 (d, J=10.8 Hz, 4H), 1.47-1.39 (m, 8H), 1.36 (s, 1H). ¹³C NMR (100 MHZ, cd₃od) δ 170.99, 154.13, 146.88, 144.92, 141.68, 140.42, 131.73, 129.51, 129.37, 128.85, 128.50, 127.98, 127.60, 122.24, 121.21, 119.63, 89.31, 71.93, 62.78, 54.48, 49.43, 49.21, 49.00, 48.79, 48.57, 48.36, 48.15, 47.83, 47.00, 40.48, 36.80, 29.95, 29.88, 29.86, 28.36, 27.32, 27.18, 25.20, 24.35, 0.57. HRMS calcd for C₃₁H₃₇N₃O₃ (MH+) 500.290769; found 500.290843.

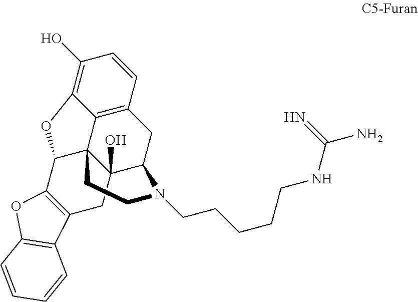

C5-Furan: ¹H NMR (399 MHz, cd₃od) δ 7.46 (t, J=7.8 Hz, 2H), 7.30 (t, J=7.4 Hz, 1H), 7.24-7.16 (m, 1H), 6.68 (s, 2H), 5.67 (s, 1H), 3.95 (d, J=6.5 Hz, 1H), 3.42 (s, 1H), 3.40-3.30 (m, 2H), 3.22 (t, J=7.0 Hz, 3H), 3.12 (td, J=12.3, 4.7 Hz, 1H), 2.93 (d, J=16.5 Hz, 1H), 2.81-2.61 (m, 2H), 2.01-1.87 (m, 2H), 1.68 (dt, J=17.3, 7.8 Hz, 4H), 1.50 (q, J=7.9 Hz, 2H). ¹³C NMR (100 MHz, cd₃od) δ 158.49, 156.81, 148.82, 144.54, 142.14, 129.49, 128.40, 126.36, 123.79, 122.17, 120.71, 120.46, 119.60, 115.29, 112.24, 83.84, 73.20, 63.17, 54.14, 47.78, 41.95, 40.18, 30.03, 29.45, 29.25, 24.85, 24.49, 24.33. HRMS calcd for C₂₈H₃₂N₄O₄ (MH+) 489.249632; found 489.249628.

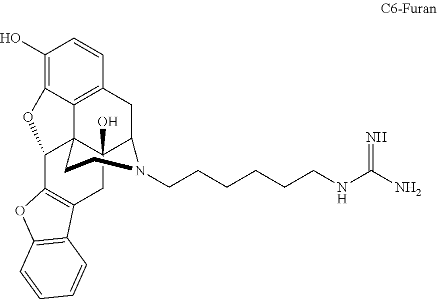

C6-Furan: ¹H NMR (399 MHz, cd₃od) δ 7.46 (t, J=8.0 Hz, 2H), 7.35-7.26 (m, 1H), 7.20 (t, J=7.5 Hz, 1H), 6.68 (s, 2H), 5.67 (s, 1H), 3.95 (d, J=6.5 Hz, 1H), 3.45-3.30 (m, 2H), 3.26-3.16 (m, 3H), 3.11 (td, J=12.4, 4.8 Hz, 1H), 3.01-2.89 (m, 2H), 2.81-2.62 (m, 2H), 1.97 (d, J=12.9 Hz, 1H), 1.87 (s, 1H), 1.64 (t, J=6.9 Hz, 1H), 1.48 (s, 1H). ¹³C NMR (100 MHz, cd₃od) δ 171.10, 158.45, 156.81, 154.44, 154.40, 148.82, 144.54, 142.13, 129.50, 128.40, 126.35, 123.79, 122.17, 120.70, 120.45, 119.59, 115.28, 112.24, 83.84, 73.20, 63.13, 54.28, 47.78, 42.11, 30.03, 29.52, 29.45, 27.10, 27.06, 25.11, 24.32. HRMS calcd for C₂₉H₃₄N₄O₄ (MH+) 503.265282; found 503.265642.

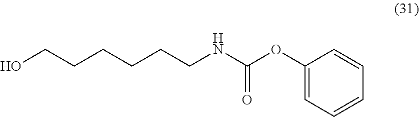 [SMILES: O=C(NCCCCCCO)Oc1ccccc1]

Preparation of C₆-urea. C₆-amidine. C₈ and C₈—OH quinoline derivatives. (31): 6-hydroxyhexamine (1.17 g, 10 mmol, 1.0 equiv.) was dissolved in anhydrous THF (40 mL) under argon and cooled to 0° C. Phenyl chloroformate (10 mmol, 1.0 equiv) was added dropwise and the mixture was allowed to warm to room temperature. After stirring for one hour, the mixture was cooled to −78° C. and quenched with the addition of few drops of 7N NH₃ in MeOH. The solvents were removed on rotary evaporator and the crude was purified on silica using 0.5% to 10% MeOH in DCM as eluent. 1.19 g, 74%. ¹H NMR (399 MHz, cdcl₃) δ 7.33 (t, J=7.7 Hz, 2H), 7.17 (t, J=7.4 Hz, 1H), 7.13-7.07 (m, 2H), 5.17 (t, J=6.0 Hz, 1H), 3.60 (t, J=6.5 Hz, 2H), 3.23 (q, J=6.7 Hz, 2H), 1.54 (p, J=6.8 Hz, 4H), 1.36 (hept, J=3.7 Hz, 4H). ¹³C NMR (100 MHz, cdcl₃) δ 155.94, 152.25, 130.46, 126.43, 122.80, 63.87, 42.26, 33.72, 30.96, 27.61, 26.52.

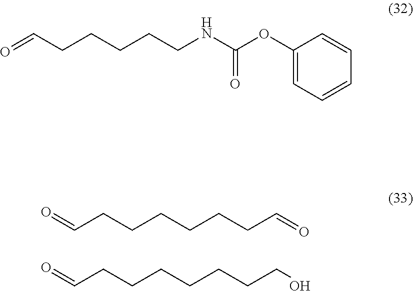 [SMILES: O=CCCCCCNC(=O)Oc1ccccc1 ; O=CCCCCCCC=O ; O=CCCCCCCCO]

(32) and (33): 1.7 g of (31) or 1.05 g of 1,8-octanediol (7.2 mmol, 1.0 equiv.) was dissolved in DCM (50 mL) containing 5 g silica and pyridinium chlorochromate (PCC, 1.54 g, 7.2 mmol, 1.0 equiv.) was added as a solution in DCM at 0° C. The mixture was then allowed to warm to room temperature and stirred for 3 hours. Excess PCC was consumed by the addition of isopropanol. The mixture was then filtered through a pad of celite, the solvent was removed on rotary evaporator and the crude product was run through a small silica column using 0.5% to 10% MeOH gradient as solvent to remove colored impurities. The mixture of aldehyde and alcohol was used without further purification.

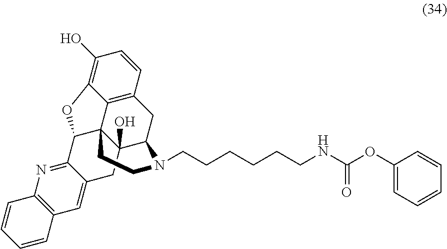

(34) was prepared using the reductive amination protocol. ¹H NMR (399 MHz, cdcl₃) δ 8.01 (d, J=8.4 Hz, 1H), 7.89 (d, J=5.3 Hz, 1H), 7.73 (d, J=8.1 Hz, 1H), 7.66 (t, J=7.6 Hz, 1H), 7.51 (t, J=7.5 Hz, 1H), 7.34 (t, J=7.6 Hz, 2H), 7.19 (q, J=7.9 Hz, 1H), 7.10 (d, J=7.9 Hz, 2H), 6.64 (q, J=8.1 Hz, 2H), 5.57 (s, 1H), 3.38-3.19 (m, 2H), 3.22 (s, 1H), 3.17 (s, 1H), 2.84 (d, J=6.2 Hz, 2H), 2.78 (d, J=16.9 Hz, 1H), 2.67 (d, J=6.9 Hz, 1H), 2.58 (t, J=7.2 Hz, 2H), 2.41 (s, 2H), 1.84 (d, J=9.6 Hz, 1H), 1.62-1.53 (m, 5H), 1.42 (d, J=6.4 Hz, 4H). ¹³C NMR (100 MHZ, cdcl₃) δ 160.53, 159.28, 156.14, 151.75, 148.43, 144.32, 142.49, 135.10, 134.81, 134.33, 133.42, 133.17, 132.71, 132.41, 132.29, 130.34, 129.11, 126.73, 124.77, 122.63, 94.89, 76.69, 67.35, 65.77, 59.36, 52.22, 48.69, 45.97, 41.02, 36.99, 34.69, 32.32, 31.88, 31.56, 28.35, 19.10.

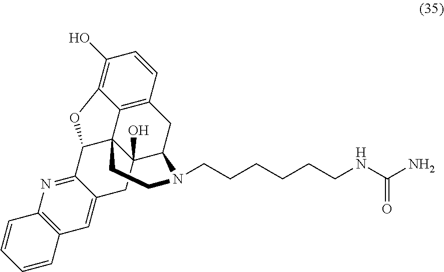

(35): 58 mg (34) (0.98 mmol, 1.0 equiv)) was dissolved in methanol (0.5 mL) and to this solution NH₄Cl (17 mg, 0.3 mmol, 3.0 equiv) 7N NH₃ in methanol (7 mL) was added. The mixture was stirred at room temperature for 7 days or until completion of the reaction and then DCM (30 mL) was added, and the solids removed by filtration. The remainder of ammonium chloride was removed by passing the crude product through a short pad of silica using 15% MeOH in DCM as eluent. ¹H NMR (399 MHz, cd₃od) δ 8.20 (s, 1H), 8.06 (d, J=8.6 Hz, 1H), 7.88 (d, J=8.2 Hz, 1H), 7.78 (ddd, J=8.5, 6.9, 1.4 Hz, 1H), 7.61 (t, J=7.6 Hz, 1H), 6.78-6.66 (m, 2H), 5.72 (s, 1H), 3.95 (d, J=6.7 Hz, 1H), 3.48 (s, 1H), 3.46-3.34 (m, 1H), 3.29-3.18 (m, 2H), 3.18-2.98 (m, 5H), 2.93 (d, J=16.6 Hz, 1H), 2.80 (td, J=13.4, 4.9 Hz, 1H), 2.04-1.95 (m, 1H), 1.86 (s, 1H), 1.69 (s, 1H), 1.57-1.42 (m, 6H). ¹³C NMR (100 MHz, cd₃od) δ 162.36, 154.12, 146.58, 145.10, 141.93, 141.24, 132.27, 129.67, 129.62, 129.24, 128.79, 128.30, 127.35, 122.49, 121.53, 119.87, 89.20, 72.13, 62.89, 54.60, 48.10, 47.24, 40.55, 36.96, 30.93, 30.15, 27.21, 27.08, 25.28, 24.57. HRMS calcd for C₃₀H₃₄N₄O₄ (MH+) 515.265282; found 515.264807.

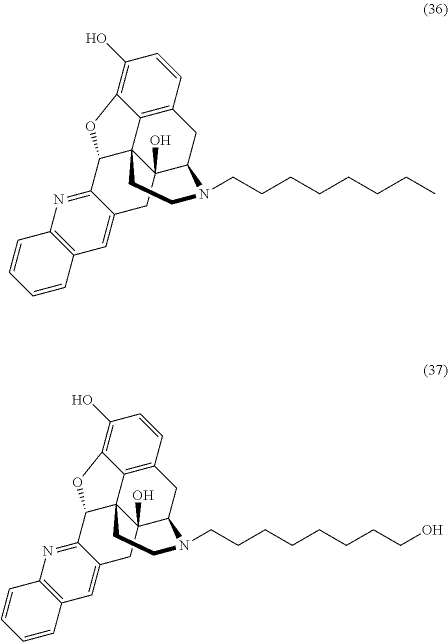

(36) and (37) were prepared as described in the generic reductive amination protocol. (36): ¹H NMR (399 MHz, cd₃od) δ 8.11 (s, 1H), 8.03 (d, J=8.6 Hz, 1H), 7.84 (d, J=8.2 Hz, 1H), 7.74 (ddd, J=8.5, 6.8, 1.4 Hz, 1H), 7.58 (dd, J=8.3, 6.8 Hz, 1H), 6.77-6.66 (m, 2H), 5.69 (s, 1H), 3.94 (d, J=6.7 Hz, 1H), 3.47 (s, 1H), 3.39 (td, J=12.2, 5.1 Hz, 1H), 3.30-3.17 (m, 2H), 3.17-2.97 (m, 3H), 2.92 (d, J=16.6 Hz, 1H), 2.79 (td, J=13.4, 5.0 Hz, 1H), 1.98 (dd, J=13.5, 3.6 Hz, 1H), 1.86 (q, J=7.9 Hz, 1H), 1.67 (s, 1H), 1.43 (m, 3H), 1.39 (dd, J=17.6, 6.3 Hz, 4H), 1.32 (p, J=4.9 Hz, 4H), 0.94-0.86 (m, 3H). ¹³C NMR (100 MHz, cd₃od) δ 154.14, 146.94, 144.93, 141.68, 140.30, 131.65, 129.53, 129.33, 128.80, 128.47, 127.93, 127.65, 122.25, 121.21, 119.63, 89.36, 71.92, 62.74, 54.51, 47.85, 47.01, 36.81, 32.70, 30.05, 30.01, 29.97, 27.40, 25.23, 24.35, 23.49, 14.2. HRMS calcd for C₃₁H₃₆N₂O₃ (MH+) 485.279869; found 485.279342.

(37): ¹H NMR (399 MHz, cd₃od) δ 8.15 (s, 1H), 8.04 (d, J=8.5 Hz, 1H), 7.86 (d, J=8.1 Hz, 1H), 7.80-7.71 (m, 1H), 7.63-7.55 (m, 1H), 6.77-6.65 (m, 2H), 5.71 (q, J=2.6 Hz, 1H), 3.95 (d, J=6.3 Hz, 1H), 3.54 (t, J=6.5 Hz, 2H), 3.48 (s, 1H), 3.39 (td, J=12.3, 5.0 Hz, 1H), 3.29-3.18 (m, 2H), 3.12 (dt, J=12.4, 6.2 Hz, 1H), 3.10-2.97 (m, 2H), 2.92 (d, J=16.7 Hz, 1H), 2.80 (td, J=13.4, 4.9 Hz, 1H), 1.98 (dd, J=13.6, 3.7 Hz, 1H), 1.85 (dt, J=12.1, 6.7 Hz, 1H), 1.67 (s, 1H), 1.53 (q, J=6.6 Hz, 2H), 1.46-1.35 (m, 8H). ¹³C NMR (100 MHz, cd₃od) δ 153.99, 146.59, 144.90, 141.70, 140.72, 131.88, 129.49, 129.36, 128.92, 128.53, 128.01, 127.33, 122.26, 121.27, 119.64, 89.13, 71.91, 62.69, 54.50, 47.85, 47.02, 40.18, 36.77, 33.34, 30.10, 30.03, 29.95, 27.31, 26.62, 25.19, 24.35. HRMS calcd for C₃₁H₃₆N₂O₄ (MH+) 501.274784; found 501.274638.

### Example 3. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

TABLE t

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A compound of Formula I: 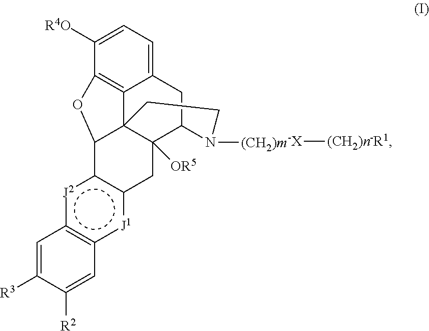
or a salt thereof;
wherein
the dotted circle of the fused monocyclic ring comprising J¹ and J² of Formula I represents aromatic bonds;
J¹ is CH or an aromatic bond joining the carbon atoms adjacent to J¹;
J² is NR^{a} wherein R^{a} is a lone pair when J¹ is CH, or R^{a} is H or (C₁-C₆)alkyl when J¹ is an aromatic bond; or
J² is O when J¹ is an aromatic bond;
R¹ is —NR^{b}(C═NR^{b})N(R^{b})₂, —OR^{b}, —N(R^{b})₂, —(C═NR^{b})N(R^{b})₂, —NR^{b}(C═O)N(R^{b})₂, —NR^{b}(C═O)OR^{b}, —NR^{b}(C═S)N(R^{b})₂, —C═NCH₂CH₂NR^{b}, —C(═NR^{b})CH₂CH₂N(R^{b})₂, or —S(═O)₂N(R^{b})₂;
R² and R³ are each independently H, halo, OR^{b}, —N(R^{b})₂, —(C₃-C₆) cycloalkyl, or —(C₁-C₆)alkyl wherein —(C₁-C₆)alkyl is optionally substituted by halo;
each R^{b} is independently H, —(C₁-C₆)alkyl, optionally substituted phenyl, or a protecting group;
R⁴ and R⁵ are each independently H, —(C₁-C₆)alkyl, or a protecting group;
X is CH₂, O, S, NR^{b}, (C═O)NR^{b}, pyrrolyl, pyrazolyl, imidazolyl, triazolyl, pyridinyl, pyrimidinyl, pyridizinyl, pyrazinyl, or phenyl;
m is an integer from 0 to 8; and
n is 5, or an integer from 0 to 8.

2. The compound of claim 1 represented by Formula II:

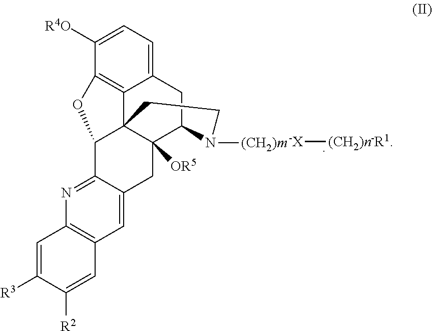

3. The compound of claim 1 represented by Formula III:

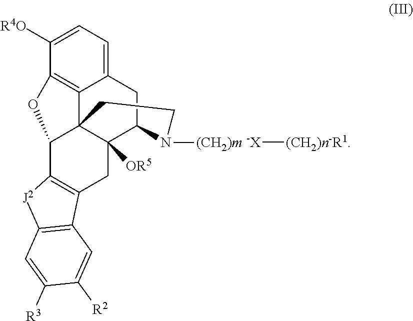

4. The compound of claim 3 wherein J² is NR^{a}.

5. The compound of claim 3 wherein J² is O.

6. The compound of claim 1 wherein R¹ is —NR^{b}(C═NR^{b})N(R^{b})₂.

7. The compound of claim 1 wherein R² and R³ are each independently H, chloro, trifluoromethyl, isopropyl, tert-butyl, cyclopropyl, methoxy, or ethoxy.

8. The compound of claim 1 wherein R⁴ and R⁵ are H.

9. The compound of claim 1 wherein R^{b} is H.

10. The compound of claim 1 wherein X is CH₂.

11. The compound of claim 1 wherein X is:

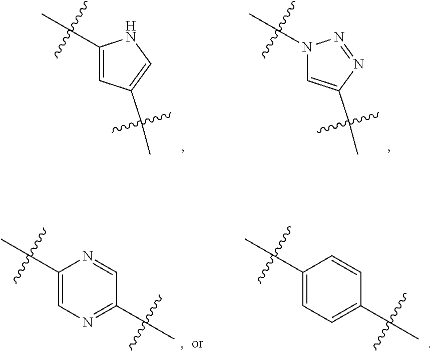 [SMILES: *c1c[nH]c(*)c1 ; *c1cn(*)nn1 ; *c1cnc(*)cn1 ; *c1ccc(*)cc1]

12. The compound of claim 1 wherein the compound is:

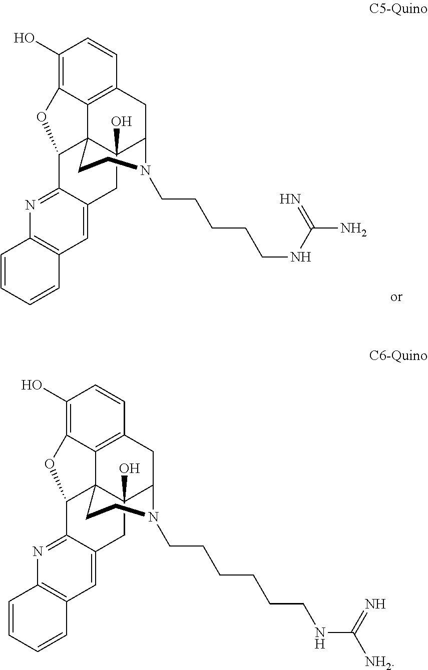

13. The compound of claim 1 wherein the compound is:

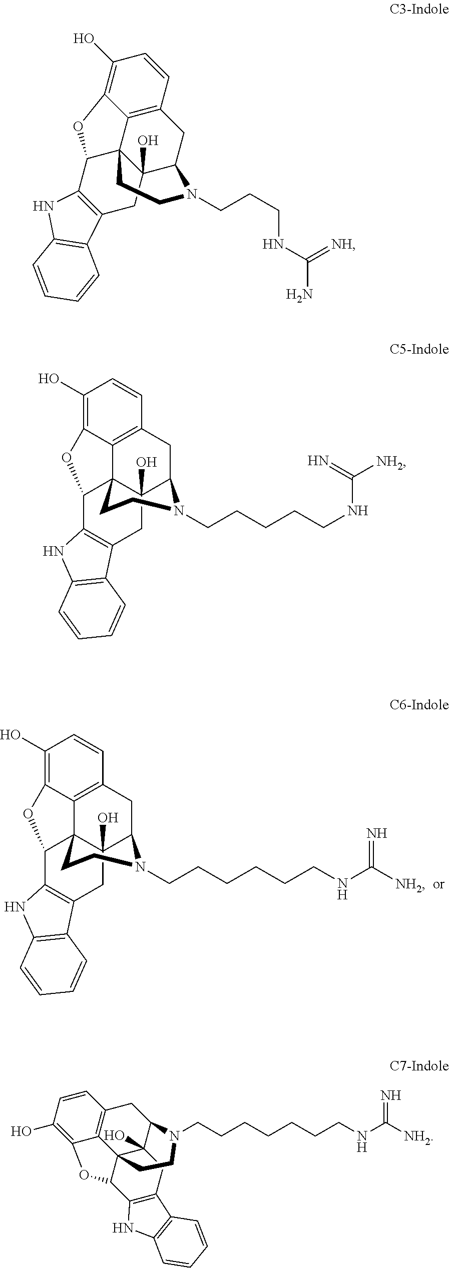

14. The compound of claim 1 wherein the compound is:

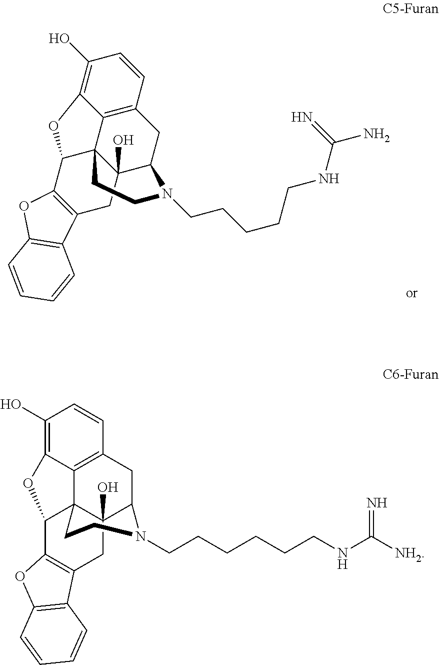

15. A method for treating pain wherein the method comprises administering to a patient having pain an effective amount of a compound or composition of claim 1, wherein pain sensed by the subject is thereby reduced or alleviated.
